SEQUENCE LISTING (10 sequences)

SEQ ID NO: 1 - protein, 17 aa, artificial sequence
  synthetic peptide
MLYLENNAQTQFSEPQC

SEQ ID NO: 2 - protein, 117 aa, artificial sequence
  synthetic peptide
QSVEESGGRLVKPDESLTLTCTVSGFSLSSYGVTWVRQAPGKGLEWIGYISHITTTYYASWAKGRFTISKTSPTTVDLKMTSLTTEDTATYFCCRGQYGSGIIYALWGPGTLVTISS

SEQ ID NO: 3 - protein, 113 aa, artificial sequence
  synthetic peptide
AQVLTQTPSPVSAAVGGTVTINCQASQSVYNNKNLAWYQQKPGQPPKLLIYYASTLASGVPSRFSGSGSGTQFTLTISGVQCDDAATYYCHGEFSCDSGDCSAFGGGTEVVVK

SEQ ID NO: 4 - protein, 10 aa, artificial sequence
  synthetic peptide
MNFETSRCAT

SEQ ID NO: 5 - protein, 10 aa, artificial sequence
  synthetic peptide
QDSDLSDPMW

SEQ ID NO: 6 - protein, 14 aa, artificial sequence
  synthetiic peptide
MLYLENNAQTQFSE

SEQ ID NO: 7 - protein, 104 aa, artificial sequence
  synthetic polypeptide
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 8 - protein, 323 aa, artificial sequence
  synthetic polypeptide
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 9 - protein, 217 aa, artificial sequence
  synthetic polypeptide
AQVLTQTPSPVSAAVGGTVTINCQASQSVYNNKNLAWYQQKPGQPPKLLIYYASTLASGVPSRFSGSGSGTQFTLTISGVQCDDAATYYCHGEFSCDSGDCSAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 10 - protein, 440 aa, artificial sequence
  synthetic polypeptide
QSVEESGGRLVKPDESLTLTCTVSGFSLSSYGVTWVRQAPGKGLEWIGYISHITTTYYASWAKGRFTISKTSPTTVDLKMTSLTTEDTATYFCCRGQYGSGIIYALWGPGTLVTISSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

(19) United States
(12) Patent Application Publication
(10) Pub. No.: US 2023/0331865 A1
(43) Pub. Date: Oct. 19, 2023

(54) ANTIBODIES FOR USE IN IMMUNOHISTOCHEMISTRY (IHC) PROTOCOLS TO DIAGNOSE CANCER

(71) Applicant: Agilent Technologies, Inc., Santa Clara, CA (US)

(72) Inventors: Morten Dræby SØRENSEN, Herlev (DK); Lasse RAMSGAARD, Herlev (DK)

(21) Appl. No.: 18/023,280

(22) PCT Filed: Aug. 25, 2021
(86) PCT No.: PCT/US2021/047521

§ 371 Date: Feb. 24, 2023

Related U.S. Application Data
(60) Provisional application No. 63/070,817, filed on Aug. 26, 2020.

(51) Int. Cl. C07K 16/30; G01N 33/58; G01N 33/574

(52) U.S. Cl. CPC C07K 16/30; G01N 33/57423; G01N 33/58; C07K 2317/20; C07K 2317/34; G01N 2474/20

(57) ABSTRACT

In alternative embodiments, provided are chimeric or recombinant rabbit antihuman p40 (p63 isoform DeltaNp63, or ΔNp63) antibodies, including products of manufacture and kits comprising them, and methods for making and using them, where the antibodies can be used for in vitro diagnostics by immunohistochemistry (IHC). In alternative embodiments, antibodies as provided herein are used in IHC protocols to diagnose a cancer, for example, a squamous-cell carcinoma (SCC) or a lung cancer such as non-small cell lung carcinoma (NCSLC) or pulmonary SCC.

### RELATED APPLICATIONS

This Patent Convention Treaty (PCT) International Patent Application claims the benefit of priority under 35 U.S.C. §119(e) to U.S. Provisional Pat. Application Serial No. (USSN) 63/070,817, Aug. 26, 2020. The aforementioned applications are expressly incorporated herein by reference in their entirety and for all purposes.

### TECHNICAL FIELD

This invention generally relates to immunohistochemistry (IHC) and cancer diagnosis. In alternative embodiments, provided are recombinant rabbit anti-human p40 (p63 isoform DeltaNp63, or ΔNp63) antibodies, including products of manufacture and kits comprising them, and methods for making and using them, where the antibodies can be used for in vitro diagnostics by immunohistochemistry (IHC). In alternative embodiments, antibodies as provided herein are used in IHC protocols to diagnose a cancer, for example, a squamous-cell carcinoma (SCC) or a lung cancer such as non-small cell lung carcinoma (NCSLC) or pulmonary SCC.

### BACKGROUND

Lung cancer is the third most common cancer worldwide. With major advances in the molecular testing of lung cancers and the introduction of targeted therapies, the distinction between adenocarcinoma and squamous cell carcinoma as well as pathologic subtyping has become important.

The protein p63 is a member of the p53 gene family. The p63 gene contains 15 exons and has a high structural and sequence homology to p53. Several p63 isoforms have been identified, many having the same alternative N-terminal of p63, compared to the canonical p63, in which the first 108 amino acids (MNFETSRCAT (SEQ ID NO:4)...QDSDLSDPMW (SEQ ID NO:5)) are substituted for MLYLENNAQTQFSE (SEQ ID NO:6). These shorter splice variants of p63, termed deltaNp63 (ΔNp63), exists in different version, all having the same alternative N-terminal. Complexity of these isoforms are generated at the COOH terminus, where splicing of exons leads to 5 different C-termini (alpha, beta, gamma, delta, and epsilon). These ΔNp63 isoforms are also collectively termed p40.

The canonical isoform of p63 contains a transactivation-competent ‘TA’ domain with homology to p53, which regulates expression of the growth-inhibitory genes. In contrast, the ΔNp63 isoforms’, or p40 proteins’, alternative N-terminal contains a transcriptionally-inactive ‘ΔN’ domain, which is thought to antagonize the activity of TAp63 and p53 (see reference 1).

Recent studies showed that expression of the protein p40, or ΔNp63 isoforms, is highly specific for squamous and basal cells and is superior to p63 for diagnosing lung squamous cell carcinoma (see reference 1). P63 can react in some cases with cellular structures in lung adenocarcinomas, which can in turn lead to misdiagnosis.

Antibodies to p40, or ΔNp63 isoforms, have been shown to aid in differentiating between lung squamous cell carcinoma and lung adenocarcinoma. Recently p40 has been described to be suitable to detect the loss of basal cells in prostate cancer with good success, and to be more specific than p63 for these cancers. It was also concluded that p40, or ΔNp63 isoforms, was the main p63 isoform in normal prostate basal cells, while the p63 expression seen in prostate carcinomas is due mainly to aberrant expression of a different p63 isoform (see reference 2).

### SUMMARY

In alternative embodiments, provided are recombinant antibodies (Abs), or antigen binding fragments thereof, or monomeric or dimeric antigen binding proteins, capable of specifically binding a human p40 (p63 isoform DeltaNp63, or ΔNp63) protein or a polypeptide, or an antigen or an epitope comprising an amino acid sequence MLYLENNAQTQFSEPQC-NH2 (SEQ ID NO:1).

In alternative embodiments, the recombinant antibody (Ab), or antigen binding fragment thereof, or monomeric or dimeric antigen binding protein, is fabricated as or in the form of:
an antigen-binding fragment (Fab, or an Ab fragment having just one constant and one variable domain of each of an Ab heavy and light chain),
a F(ab′)₂ (or an Ab digested by pepsin yielding two fragments: a F(ab′)₂ fragment and a pFc′ (pepsin cleavage Fc) fragment),
a Fab′ (a single chain of a F(ab′)₂ fragment),
a single-chain variable fragment (scFv) (or a fusion protein of a variable region of an Ab heavy and light chain connected together with a linker peptide optionally of about ten to about 25 amino acids in length),
a (scFv)₂, or a di-scFv or a bi-scFv, or a single peptide chain having two variable heavy and two variable light regions yielding tandem scFv,
a minibody (or a fusion protein of a variable region of an Ab heavy and light chain connected together with an alkyl group, optionally a methyl or an ethyl group)
a diabody (or an scFv with a linker peptide too short (optionally about five amino acids) for the two variable regions to fold together forcing the scFvs to dimerize), a triabody or a tetrabody (or an scFv with a linker peptide too short (optionally about one or two amino acids) for the two variable regions to fold together forcing the scFvs to trimerize or tetramize),
a single-domain antibody (dAB) (or a single variable region of an Ab heavy or Ab light chain),
a plurality of complementarity determining region (CDR) fragments, or
a multispecific antibody formed from two or more antibody fragments.

In alternative embodiments, the antibody or dimeric antigen binding protein comprises a heavy chain variable region paired with or bound to a light chain variable region such that the antibody or the dimeric antigen binding protein is capable of specifically binding a human p40 (p63 isoform DeltaNp63, or ΔNp63) protein or polypeptide, an antigen or an epitope comprising an amino acid sequence MLYLENNAQTQFSEPQC-NH2 (SEQ ID NO:1).

In alternative embodiments, of the recombinant antibody (Ab), or antigen binding fragment thereof, or monomeric or dimeric antigen binding protein: the Ab, or antigen binding fragment thereof, or monomeric or dimeric antigen binding protein, comprises:
(a) a heavy chain variable region (VH) capable of specifically binding a human p40 (p63 isoform DeltaNp63, or ΔNp63) protein or polypeptide, an antigen or an epitope comprising an amino acid sequence MLYLENNAQTQFSEPQC-NH2 (SEQ ID NO:1), comprising:
   (1) an amino acid sequence comprising the three CDR1, CDR2 and CDR3 complementarity determining regions (CDRs) of SEQ ID NO:1, or CDR1 amino acid (aa) residues GFSLSSYG (residues 25 to 32 of SEQ ID NO:2), CDR2 aa residues ISHITTT (residues 50 to 56 of SEQ ID NO:2), and CDR3 aa residues CRGQYGSGIIYAL (residues 94 to 106 of SEQ ID NO:2), or
   (2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:2, or CDR1 amino acid (aa) residues GFSLSSYG (residues 25 to 32 of SEQ ID NO:2), CDR2 aa residues ISHITTT (residues 50 to 56 of SEQ ID NO:2), and CDR3 aa residues CRGQYGSGIIYAL (residues 94 to 106 of SEQ ID NO:2), or
   (3) an amino acid sequence having at least about 70%, 75%, 80%, 85%, 90%, 95%, 98% sequence identity, or between about 70% to 100% sequence identity, to SEQ ID NO:2, or an amino acid sequence having complete sequence identity to SEQ ID NO:2;
(b) a light chain variable region (VL) capable of specifically binding a human p40 (p63 isoform DeltaNp63, or ΔNp63) protein or polypeptide, an antigen or an epitope comprising an amino acid sequence MLYLENNAQTQFSEPQC-NH2 (SEQ ID NO:1), comprising:
   (1) an amino acid sequence comprising the three CDR1, CDR2 and CDR3 complementarity determining regions (CDRs) of SEQ ID NO:3, or CDR1 amino acid (aa) residues QSVYNNKN (residues 27 to 34 of SEQ ID NO:3), CDR2 aa residues YAS (residues 52 to 54 of SEQ ID NO:3), and CDR3 aa residues HGEFSCDSGDCSA (residues 91 to 103 of SEQ ID NO:3), or
   (2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:3, or CDR1 amino acid (aa) residues QSVYNNKN (residues 27 to 34 of SEQ ID NO:3), CDR2 aa residues YAS (residues 52 to 54 of SEQ ID NO:3), and CDR3 aa residues HGEFSCDSGDCSA (residues 91 to 103 of SEQ ID NO:3); or
   (3) an amino acid sequence having at least about 70%, 75%, 80%, 85%, 90%, 95%, 98% sequence identity, or between about 70% to 100% sequence identity, to SEQ ID NO:3, or an amino acid sequence having complete (100%) sequence identity to SEQ ID NO:3; or
(c) a heterodimer capable of specifically binding a human p40 (p63 isoform DeltaNp63, or ΔNp63) protein or polypeptide, an antigen or an epitope comprising an amino acid sequence MLYLENNAQTQFSEPQC-NH2 (SEQ ID NO:1), comprising the heavy chain variable region (VH) of (a) and the light chain variable region (VL) of (b).

In alternative embodiments, of the recombinant antibody (Ab), or antigen binding fragment thereof, or monomeric or dimeric antigen binding protein: the heavy chain variable region comprises an amino acid sequence:

QSVEESGGRLVKPDESLTLTCTVSGFSLSSYGVTWVRQAPGKGLEWIGYI SHITTTYYASWAKGRFTISKTSPTTVDLKMTSLTTEDTATYFCCRGQYGS GIIYALWGPGTLVTISS (SEQ ID NO:2)

, or

SEQ ID NO:2 having one or more amino acid substitutions or deletions, and the recombinant antibody (Ab), or antigen binding fragment thereof, or monomeric or dimeric antigen binding protein retains its ability to specifically bind to a peptide or epitope comprising SEQ ID NO:1, or a fragment of a polypeptide of SEQ ID NO:1, a p40 (or p63 isoform DeltaNp63) polypeptide or peptide or a fragment of a p40 polypeptide or peptide.

In alternative embodiments, the light chain variable region comprises an amino acid sequence:

AQVLTQTPSPVSAAVGGTVTINCQASQSVYNNKNLAWYQQKPGQPPKLLI
YYASTLASGVPSRFSGSGSGTQFTLTISGVQCDDAATYYCHGEFSCDSGD CSAFGGGTEVVVK (SEQ ID NO:3), or

SEQ ID NO:3 having one or more amino acid substitutions or deletions, and the recombinant antibody (Ab), or antigen binding fragment thereof, or monomeric or dimeric antigen binding protein retains its ability to specifically bind to a peptide or epitope comprising SEQ ID NO:1, or a fragment of a polypeptide of SEQ ID NO:1, a p40 (or p63 isoform DeltaNp63) polypeptide or peptide or a fragment of a p40 polypeptide or peptide.

In alternative embodiments, the heavy chain variable region comprises an amino acid sequence:

QSVEESGGRLVKPDESLTLTCTVSGFSLSSYGVTWVRQAPGKGLEWIGYI SHITTTYYASWAKGRFTISKTSPTTVDLKMTSLTTEDTATYFCCRGQYGS GIIYALWGPGTLVTISS (SEQ ID NO:2)

, or

SEQ ID NO:2 having one or more amino acid substitutions or deletions, and the recombinant antibody (Ab), or antigen binding fragment thereof, or monomeric or dimeric antigen binding protein retains its ability to specifically bind to a peptide or epitope comprising SEQ ID NO:1, or a fragment of a polypeptide of SEQ ID NO:1, a p40 (or p63 isoform DeltaNp63) polypeptide or peptide or a fragment of a p40 polypeptide or peptide.

In alternative embodiments, the light chain variable region comprises an amino acid sequence:

AQVLTQTPSPVSAAVGGTVTINCQASQSVYNNKNLAWYQQKPGQPPKLLI
YYASTLASGVPSRFSGSGSGTQFTLTISGVQCDDAATYYCHGEFSCDSGD CSAFGGGTEVVVK (SEQ ID NO:3)

, or

SEQ ID NO:3 having one or more amino acid substitutions or deletions, and the recombinant antibody (Ab), or antigen binding fragment thereof, or monomeric or dimeric antigen binding protein retains its ability to specifically bind to a peptide or epitope comprising SEQ ID NO:1, or a fragment of a polypeptide of SEQ ID NO:1, a p40 (or p63 isoform DeltaNp63) polypeptide or peptide or a fragment of a p40 polypeptide or peptide.

In alternative embodiments, the heavy chain variable region comprises an amino acid sequence:

QSVEESGGRLVKPDESLTLTCTVSGFSLSSYGVTWVRQAPGKGLEWIGYI SHITTTYYASWAKGRFTISKTSPTTVDLKMTSLTTEDTATYFCCRGQYGS GIIYALWGPGTLVTISS (SEQ ID NO:2)

, or

SEQ ID NO:2 having one or more amino acid substitutions or deletions, and the recombinant antibody (Ab), or antigen binding fragment thereof, or monomeric or dimeric antigen binding protein retains its ability to specifically bind to a peptide or epitope comprising SEQ ID NO:1, or a fragment of a polypeptide of SEQ ID NO:1, a p40 (or p63 isoform DeltaNp63) polypeptide or peptide or a fragment of a p40 polypeptide or peptide, and

the light chain variable region comprises an amino acid sequence:

AQVLTQTPSPVSAAVGGTVTINCQASQSVYNNKNLAWYQQKPGQPPKLLI
YYASTLASGVPSRFSGSGSGTQFTLTISGVQCDDAATYYCHGEFSCDSGD CSAFGGGTEVVVK (SEQ ID NO:3)

, or

SEQ ID NO:3 having one or more amino acid substitutions or deletions, and the recombinant antibody (Ab), or antigen binding fragment thereof, or monomeric or dimeric antigen binding protein retains its ability to specifically bind to a peptide or epitope comprising SEQ ID NO:1, or a fragment of a polypeptide of SEQ ID NO:1, a p40 (or p63 isoform DeltaNp63) polypeptide or peptide or a fragment of a p40 polypeptide or peptide.

In alternative embodiments, the light chain variable region further comprises at least a portion of a light chain constant region.

In alternative embodiments, the light chain constant region comprises an amino acid sequence:

GDPVAPTVLIFPPAADQVATGTVTIVCVANKYFPDVTVTWEVDGTTQTTG
IENSKTPQNSADCTYNLSSTLTLTSTQYNSHKEYTCKVTQGTTSVVQSFN
RGDC(SEQ ID NO:7).

In alternative embodiments, the heavy chain variable region further comprises at least a portion of a heavy chain constant region.

In alternative embodiments, the heavy chain constant region comprises an amino acid sequence:

GQPKAPSVFPLAPCCGDTPSSTVTLGCLVKGYLPEPVTVTWNSGTLTNGV
RTFPSVRQSSGLYSLSSVVSVTSSSQPVTCNVAHPATNTKVDKTVAPSTC
SKPTCPPPELLGGPSVFIFPPKPKDTLMISRTPEVTCVVVDVSQDDPEVQ
FTWYINNEQVRTARPPLREQQFNSTIRVVSTLPIAHQDWLRGKEFKCKVH
NKALPAPIEKTISKARGQPLEPKVYTMGPPREELSSRSVSLTCMINGFYP
SDISVEWEKNGKAEDNYKTTPAVLDSDGSYFLYSKLSVPTSEWQRGDVFT
CSVMHEALHNHYTQKSISRSPGK(SEQ ID NO:8).

In alternative embodiments, the light chain variable region further comprises at least a portion of a light chain constant region; and, the heavy chain variable region further comprises at least a portion of a heavy chain constant region.

In alternative embodiments, the light chain comprises a variable region as set forth in SEQ ID NO:3, and a constant region as set forth in SEQ ID NO:7, or

AQVLTQTPSPVSAAVGGTVTINCQASQSVYNNKNLAWYQQKPGQPPKLLI
YYASTLASGVPSRFSGSGSGTQFTLTISGVQCDDAATYYCHGEFSCDSGD
CSAFGGGTEVVVKGDPVAPTVLIFPPAADQVATGTVTIVCVANKYFPDVT
VTWEVDGTTQTTGIENSKTPQNSADCTYNLSSTLTLTSTQYNSHKEYTCK
VTQGTTSVVQSFNRGDC (SEQ ID NO:9).

In alternative embodiments, the heavy chain comprises a variable region as set forth in SEQ ID NO:2, and a constant region as set forth in SEQ ID NO:8, or

QSVEESGGRLVKPDESLTLTCTVSGFSLSSYGVTWVRQAPGKGLEWIGYI
SHITTTYYASWAKGRFTISKTSPTTVDLKMTSLTTEDTATYFCCRGQYGS
GIIYALWGPGTLVTISSGQPKAPSVFPLAPCCGDTPSSTVTLGCLVKGYL
PEPVTVTWNSGTLTNGVRTFPSVRQSSGLYSLSSVVSVTSSSQPVTCNVA
HPATNTKVDKTVAPSTCSKPTCPPPELLGGPSVFIFPPKPKDTLMISRTP
EVTCVVVDVSQDDPEVQFTWYINNEQVRTARPPLREQQFNSTIRVVSTLP
IAHQDWLRGKEFKCKVHNKALPAPIEKTISKARGQPLEPKVYTMGPPREE
LSSRSVSLTCMINGFYPSDISVEWEKNGKAEDNYKTTPAVLDSDGSYFLY
SKLSVPTSEWQRGDVFTCSVMHEALHNHYTQKSISRSPGK(SEQ ID NO
:10).

In alternative embodiments, provided is an antibody, or a heterodimeric protein, having a light chain as set forth in SEQ ID NO:9, and a heavy chain as set forth in SEQ ID NO:10.

In alternative embodiments, the heavy chain constant region comprises amino acid sequence from a IgG, IgM, IgA, IgD or IgE isotype; or the light chain constant region comprises amino acid sequence from a kappa (κ) or lambda (λ) isotype.

In alternative embodiments, the at least a portion of the heavy chain constant region, at least a portion of the light chain constant region, or at least a portion of the heavy chain constant region and the light chain constant region, is or comprises amino acid sequence of a human, a rabbit, a mouse or a rat origin or comprises constant region amino acid sequence derived from a human, a rabbit, a mouse or a rat.

In alternative embodiments, at least a portion of the heavy chain constant region, at least a portion of the light chain constant region, or at least a portion of the heavy chain constant region and the light chain constant region, is or comprises a synthetic amino acid sequence.

In alternative embodiments, the recombinant antibody, the antigen binding fragment thereof, or monomeric or dimeric antigen binding protein, or the heavy chain constant region, or the light chain constant region, or the heavy chain constant region and the light chain constant region, further comprises or is bound to a detectable agent or a binding moiety.

In alternative embodiments, the detectable agent or binding moiety is covalently conjugated to the recombinant antibody (Ab), or antigen binding fragment thereof, or monomeric or dimeric antigen binding protein.

In alternative embodiments, the detectable agent or binding moiety comprises a biotin, a fluorescent or chemiluminescent label, a fluorophore, sulfoindocyanine, nile red, rhodamine, perylene, fluorenyl, coumarin, 7-methoxycoumarin (Mca), dabcyl, [2-(4-nitro-2,1,3-benzoxadiazol-7-yl)aminoethyl]trimethylammonium (NBD), Nile blue, Tamra, boron-dipyrromethene (BODIPY), or derivatives thereof, a dye, a radioisotope, a quantum dot or photoluminescent aqueous nanocrystal, a hapten, or an antibody binding epitope or domain.

In alternative embodiments, the fluorophore is or comprises a dansyl, a fluorescein, a carboxyfluorescein (FAM) or a 6-FAM moiety. In alternative embodiments, the dye is or comprises a cyanine dye, a Cy3 or a Cy5. In alternative embodiments, the hapten is or comprises a biotin, a theophylline, a digoxigenin, a carborane, a fluorescein or a bromodeoxyuridine moiety.

In alternative embodiments, provided are chimeric or recombinant nucleic acids comprising: a nucleic acid sequence encoding a chimeric or recombinant antibody or dimeric antigen binding protein as provided herein; or, a nucleic acid sequence comprising SEQ ID NO:2 or SEQ ID NO:3, wherein optionally the chimeric or recombinant nucleic acid further comprises and is operatively linked to a transcriptional regulatory element, and optionally the transcriptional regulatory element comprises a promoter, and optionally the promoter is an inducible promoter or a constitutive promoter.

In alternative embodiments, provided are expression cassettes, vectors, recombinant viruses, artificial chromosomes, cosmids or plasmids comprising a chimeric or a recombinant nucleic acid as provided herein.

In alternative embodiments, provided are cells comprising a chimeric or recombinant antibody or dimeric antigen binding protein as provided herein, a chimeric or recombinant nucleic acid as provided herein, or an expression cassette, vector, recombinant virus, artificial chromosome, cosmid or plasmid as provided herein, wherein optionally the cell is a bacterial, fungal, mammalian, yeast, insect or plant cell.

In alternative embodiments, provided are methods for detecting the presence of a human p40 protein in a cell, a tissue, an organ or a portion of any of the foregoing, comprising:
(a) contacting the cell, tissue or organ or portion of any of the foregoing with a chimeric or recombinant antibody or dimeric antigen binding protein as provided herein, and
(b) detecting binding of the chimeric or recombinant antibody or dimeric antigen binding protein with a p40 polypeptide or a MLYLENNAQTQFSEPQC-NH2 (SEQ ID NO: 1)-comprising polypeptide in the cell, tissue or organ or portion of any of the foregoing,
thereby detecting the presence of a human p40 protein in a cell, a tissue, an organ or a portion of any of the foregoing, comprising contacting the cell, tissue or organ or portion of any of the foregoing.

In alternative embodiments of methods as provided herein:
the contacting comprises use of an immunohistochemistry (IHC) assay;
the method further comprises contacting the chimeric or recombinant antibody or dimeric antigen binding protein with a detectable agent to indicate or signal binding of the chimeric or recombinant antibody or dimeric antigen binding protein to the human p40 protein; or
the detectable agent specifically binds to the chimeric or recombinant antibody or dimeric antigen binding protein.

In alternative embodiments, provided are methods for detecting or diagnosing a cancer, wherein optionally the cancer is: a lung cancer, a lung squamous cell carcinoma, a lung adenocarcinoma (adenocarcinoma of the lung), a non-small-cell lung cancer, an adenocarcinoma, or a squamous cell carcinoma, wherein the method comprises detection of expression or presence of a human p40 protein in a cell, tissue or organ sample, optionally in a cell, tissue or organ sample from an individual in need thereof, using a chimeric or recombinant antibody as provided herein to detect the expression or presence of the human p40 protein in the cell, tissue or organ sample, and detecting the expression or presence of the human p40 protein in the cell, tissue or organ sample detects or diagnoses the cancer. In alternative embodiments, wherein the detection comprises conducting an immunohistochemistry (IHC) assay.

In alternative embodiments, provided are methods for treating, ameliorating or preventing a cancer comprising first detecting or diagnosing the cancer using a method as provided herein, followed by treatment of the individual in need thereof for the treatment, amelioration or prevention of the cancer. In alternative embodiments, the cancer is a lung cancer, a lung squamous cell carcinoma, a lung adenocarcinoma (adenocarcinoma of the lung), a non-small-cell lung cancer, an adenocarcinoma, or a squamous cell carcinoma.

In alternative embodiments, provided are uses of a chimeric or recombinant antibody as provided herein for detecting or diagnosing a cancer, or treating, ameliorating or preventing a cancer, wherein optionally the use comprises use of an immunohistochemistry (IHC) assay. In alternative embodiments of the uses the cancer is a lung cancer, a lung squamous cell carcinoma, a lung adenocarcinoma (adenocarcinoma of the lung), a non-small-cell lung cancer, an adenocarcinoma, or a squamous cell carcinoma.

In alternative embodiments, provided are chimeric or recombinant antibodies as provided herein for use in detecting or diagnosing a cancer, or treating, ameliorating or preventing a cancer, wherein optionally the use comprises use of an immunohistochemistry (IHC) assay. In alternative embodiments, the cancer is a lung cancer, a lung squamous cell carcinoma, a lung adenocarcinoma (adenocarcinoma of the lung), a non-small-cell lung cancer, an adenocarcinoma, or a squamous cell carcinoma.

In alternative embodiments, provided are kits comprising a chimeric or recombinant antibody as provided herein, or an expression cassette, vector, recombinant virus, artificial chromosome, cosmid or plasmid as provided herein. In alternative embodiments, the kit comprises (or further comprises) components needed for an immunohistochemistry (IHC) assay, or optionally comprises instructions for practicing a method as provided herein.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1, FIG. 2, FIG. 3 and FIG. 4, graphically illustrate results of an ELISA detecting anti-p40 antibodies in plasma from rabbits immunized with the p40 peptide SEQ ID NO:1, where in the graph optical density OD is a function of plasma dilutions, as discussed in detail in Example 1, below.

FIG. 5 illustrates a gel image showing the results of PCR reactions on cDNA derived from B-cell cultures to amplify the heavy and light variable domains; PCR-amplified VH and VL domains of 4 selected clones are shown, as discussed in detail in Example 1, below.

FIG. 6 illustrates Table 1, showing data from an ELISA-based reactivity analysis of re-cloned rabbit B-cell clones derived from B-cell selection and culture against the target P40 biotin peptide MS1891.2, as discussed in detail in Example 1, below.

FIG. 7 illustrates Table 2, showing data from an ELISA-based reactivity analysis of re-cloned rabbit B-cell clones derived from B-cell selection and culture against an irrelevant biotinylated peptide, as discussed in detail in Example 1, below.

FIG. 8 illustrates an image of clone 15F11 antibodies staining squamous epithelial cells of tonsil, as discussed in detail in Example 1, below.

FIG. 9 illustrates an image of clone 15F11 antibodies staining squamous epithelial cells of tonsil, as discussed in detail in Example 1, below.

FIG. 10 illustrates an image of clone 15F11 antibodies staining cytotrophoblasts of placenta, as discussed in detail in Example 1, below.

FIG. 11 illustrates an image of clone 15F11 antibodies staining cytotrophoblasts of placenta, as discussed in detail in Example 1, below.

FIG. 12 illustrates an image of clone 15F11 antibodies staining lung squamous cell carcinoma, as discussed in detail in Example 1, below.

FIG. 13 illustrates an image of clone 15F11 antibodies staining lung adenocarcinoma, as discussed in detail in Example 1, below.

FIG. 14 illustrates an image of anti-p40 clone 15F11, and BC28 antibodies staining esophagus (100X image), as discussed in detail in Example 1, below.

FIG. 15 illustrates an image of anti-p40 clone 15F11, and BC28 antibodies staining placenta (100X image) under various conditions, as discussed in detail in Example 1, below.

FIG. 16 illustrates an image of anti-p40 clone 15F11, and BC28 antibodies staining tonsil, as discussed in detail in Example 1, below.

FIG. 17 illustrates an image of anti-p40 clone 15F11, and BC28 antibodies staining tonsil, as discussed in detail in Example 1, below.

FIG. 18 illustrates an image of anti-p40 clone 15F11, and BC28 antibodies staining normal prostate (100X image) under various conditions, as discussed in detail in Example 1, below.

FIG. 19 illustrates an image of anti-p40 clone 15F11, BC28 antibodies staining kidney (100X image), as discussed in detail in Example 1, below.

FIG. 20 illustrates an image of anti-p40 clone 15F11, BC28 antibodies staining appendix (100X image), as discussed in detail in Example 1, below.

FIG. 21 illustrates a first image of anti-p40 clone 15F11, BC28 antibodies staining lung squamous cell carcinoma (100X image) under various conditions, as discussed in detail in Example 1, below.

FIG. 22 illustrates a second image of anti-p40 clone 15F11, BC28 antibodies staining lung squamous cell carcinoma (100X image) under various conditions, as discussed in detail in Example 1, below.

FIG. 23 illustrates a first image of anti-p40 clone 15F11, BC28 antibodies staining lung adenocarcinoma (100X image) under various conditions, as discussed in detail in Example 1, below.

FIG. 24 illustrates a second image of anti-p40 clone 15F11, BC28 antibodies staining lung adenocarcinoma (100X image) under various conditions, as discussed in detail in Example 1, below.

FIG. 25 illustrates an image of anti-p40 clone 15F11, BC28 antibodies staining lung large cell carcinoma (100X image) under various conditions, as indicated in the figure.

FIG. 26 illustrates an image of anti-p40 clone 15F11, BC28 antibodies staining mamma ductal carcinoma (100X image) under various conditions, as discussed in detail in Example 1, below.

FIG. 27 illustrates an image of anti-p40 clone 15F11, BC28 antibodies staining pancreatic adenocarcinoma (100X image) under various conditions, as discussed in detail in Example 1, below.

FIG. 28 illustrates an image of anti-p40 clone 15F11, BC28 antibodies staining diffuse large B-cell lymphoma (DLBCL) (100X image) under various conditions, as discussed in detail in Example 1, below.

FIG. 29 Positive normal tonsil (p40 High Expressing (HE) and p40 Low Expression (LE)) and negative control tissue tested with anti-p40 clone 15F11, as discussed in detail in Example 1, below.

FIG. 30 Lung squamous cell carcinoma (clin pos) and negative lung adenocarcinoma (clin neg) tissues were tested with two-fold dilutions of the antibody, as discussed in detail in Example 1, below.

FIG. 31 illustrates TABLE 3, showing data from IHC used to test the exemplary recombinant rabbit monoclonal anti-human p40 antibodies on a panel of cancer cells and tissues, as discussed in detail in Example 1, below.

FIG. 32 illustrates TABLE 4, showing data from staining with anti-p40 clone 15F11 as well as a negative control reagent, as discussed in detail in Example 1, below.

Like reference symbols in the various drawings indicate like elements.

### DETAILED DESCRIPTION

In alternative embodiments, provided are recombinant rabbit anti-human p40 (p63 isoform DeltaNp63, or ΔNp63) antibodies (Abs), including products of manufacture and kits comprising them, and methods for making and using them, where the antibodies can be used for in vitro diagnostics by immunohistochemistry (IHC). In alternative embodiments, antibodies as provided herein are used in IHC protocols to diagnose a cancer, for example, a squamous-cell carcinoma (SCC) or a lung cancer such as non-small cell lung carcinoma (NCSLC) or pulmonary SCC.

Rabbit anti-human p40 antibodies as provided herein were developed by B-cell selection using the peptide antigen: P40 (1-16), MLYLENNAQTQFSEPQC-NH2 (SEQ ID NO:1). Serum samples of rabbits immunized using this peptide were evaluated by immunohistochemistry (IHC). Post-immunization B-cells were isolated from the rabbit and supernatant from thousands of clones were screened using enzyme-linked immunosorbent assays (ELISA). Supernatants of ELISA positive clones were evaluated by IHC. Four clones were chosen for sequencing, after which the nucleic acids encoding the antibodies were synthesized and inserted into expression vectors based on a pTT5 backbone. These expression vectors were used in human embryonic kidney 293 cells (HEK) cells for generation of recombinant antibody. A clone designated “15F11” was chosen as best performing clone:

### Sequence of Antibody Clone 15F11

### 15F11 Heavy Chain Variable Region:

QSVEESGGRLVKPDESLTLTCTVSGFSLSSYGVTWVRQAPGKGLEWIGYI SHITTTYYASWAKGRFTISKTSPTTVDLKMTSLTTEDTATYFCCRGQYGS GIIYALWGPGTLVTISS (SEQ ID NO:2)

CDR regions are highlighted in bold and are defined according to IMGT (ImMunoGeneTics, Laboratoire d′ImmunoGénétique Moléculaire (LIGM)) numbering: CDR1 aa 25 to 32, CDR2 aa 50 to 56, and CDR3 aa 94 to 106.

### 15F11 Light Chain Variable Region:

AQVLTQTPSPVSAAVGGTVTINCQASQSVYNNKNLAWYQQKPGQPPKLLI
YYASTLASGVPSRFSGSGSGTQFTLTISGVQCDDAATYYCHGEFSCDSGD CSAFGGGTEVVVK (SEQ ID NO:3)

CDR regions are highlighted in bold and are defined according to IMGT numbering: CDR1 aa 27 to 34, CDR2 aa 52 to 54, and CDR3 aa 91 to 103.

In alternative embodiments, the exemplary recombinant anti-human p40 Abs or dimeric antigen binding proteins comprising heavy chain variable region SEQ ID NO:2 and light chain variable region SEQ ID NO:3 are each bound to or fused (or only one is bound or fused) to an immunoglobulin heavy and light chain constant region, respectively, which can be for example, of human, rabbit, mouse or rat origin, or can be partially or entirely synthetic. The heavy and/or light chains can be of any isotype, for example, the heavy chain can comprise amino acid sequence from a IgG, IgM, IgA, IgD or IgE isotype; or the light chain can comprise amino acid sequence from a kappa (κ) or lambda (λ) isotype.

In alternative embodiments, exemplary recombinant anti-human p40 Abs, or dimeric antigen binding proteins, comprising heavy chain variable region SEQ ID NO:2 and light chain variable region SEQ ID NO:3, are (or are configured or assembled as) antibody (Ab) fragments, including for example, Abs or dimeric antigen binding proteins as provided herein in the form of Fab, Fab′, F(ab′)₂, scFv, (scFv)₂, dAb, and complementarity determining region (CDR) fragments, linear antibodies, single-chain antibody molecules, minibodies, diabodies, and multispecific antibodies formed from antibody fragments.

In alternative embodiments, exemplary recombinant anti-human p40 Abs, or dimeric antigen binding proteins, comprising heavy chain variable region SEQ ID NO:2 and light chain variable region SEQ ID NO:3, are (or are configured or assembled as) Fv fragments, i.e., as an antibody fragment which contains a complete antigen recognition and binding site, including a dimer of one heavy and one light chain variable domain in tight association, which can be covalent in nature, for example as an scFv. It is in this configuration that the three CDRs of each variable domain interact to define an antigen binding site on the surface of the V_{H}-V_{L} dimer. The six CDRs or a subset thereof can confer antigen binding specificity to the antibody. In one embodiment, a single variable domain, or half of an Fv comprising only three CDRs specific for an antigen, has the ability to recognize and bind antigen, although usually at a lower affinity than the entire binding site.

In alternative embodiments, exemplary recombinant anti-human p40 Abs, or dimeric antigen binding proteins, comprising heavy chain variable region SEQ ID NO:2 and light chain variable region SEQ ID NO:3, are (or are configured or assembled as) F(ab′)₂ or Fab fragments, which contain a variable and constant domain of a light chain and a variable domain and the first constant domain (CH1) of a heavy chain. F(ab′)₂ antibody fragments comprise a pair of Fab fragments which are linked, for example, covalently linked, near their carboxy termini, for example, by hinge cysteines or equivalents, between them. In alternative embodiments, any chemical coupling of antibody fragments known in the art can be used.

In alternative embodiments, exemplary recombinant anti-human p40 Abs, or dimeric antigen binding proteins, comprising heavy chain variable region SEQ ID NO:2 and light chain variable region SEQ ID NO:3, are (or are configured or assembled as) single-chain Fv or scFv antibody fragments, which can comprise the V_{H} and V_{L} domains of antibody, wherein these domains are present in a single polypeptide chain. In alternative embodiments, Fv polypeptides as provided herein further comprise a polypeptide linker between the V_{H} and V_{L} domains, which enables the scFv to form the desired structure for antigen binding.

In alternative embodiments, exemplary recombinant anti-human p40 Abs, or dimeric antigen binding proteins, comprising heavy chain variable region SEQ ID NO:2 and light chain variable region SEQ ID NO:3, are (or are configured or assembled as) diabodies, i.e., as small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites.

In alternative embodiments, exemplary recombinant anti-human p40 Abs, or dimeric antigen binding proteins, comprising heavy chain variable region SEQ ID NO:2 and light chain variable region SEQ ID NO:3, are (or are configured or assembled as) linear antibodies, for example, as antibodies described in Zapata et al. (1995 Protein Eng, 8(10):1057-1062). In alternative embodiments, linear antibodies as provided herein comprise a pair of tandem Fd segments (V_{H}—C_{H1}—V_{H}—C_{H1}) which, together with complementary light chain polypeptides, form a pair of antigen binding regions. In alternative embodiments, linear antibodies as provided herein are bispecific or monospecific.

### Expression of Recombinant Antibodies

In alternative embodiments, recombinant antibodies (Abs) or antigen binding fragments thereof, or monomeric or dimeric antigen binding proteins as provided herein, including the exemplary recombinant anti-human p40 Abs comprising heavy chain variable region SEQ ID NO:2 and light chain variable region SEQ ID NO:3, any of which may or may not have a signal peptide or other heterologous peptide or tag, are expressed as a recombinant polypeptide or Ab using any expression vehicle or expression system, for example, a vector such as a viral vector, a phage, a plasmid or a cosmid. In alternative embodiments, a constant heavy chain is expressed together with a light chain, for example, a heavy chain can be expressed with a kappa-1 light chain.

In alternative embodiments, nucleic acids encoding the respective heavy and light chains, or the heavy chain and the light chain, are encoded in separate expression vehicles, or in the same expression vehicle or expression system.

In some embodiments, the recombinant antibodies (Abs) or antigen binding fragments thereof, or monomeric or dimeric antigen binding proteins as provided herein, including the heavy and light chains can be (cis- or trans-) as provided herein, are expressed from a pTT5™ vector(s) (National Research Council Canada, NRC-CNRC, Canada) or equivalents.

In alternative embodiments, the expression vehicles (such as a vector, plasmid virus or phage) containing nucleic acids encoding recombinant antibodies (Abs) or antigen binding fragments thereof, or monomeric or dimeric antigen binding proteins as provided herein are expressed in in vitro expression systems or are expressed in cultured tissues or cells, for example, they are expressed in a human embryonic kidney (HEK) cell such as an HEK293-6E cell.

In alternative embodiment, the expression vehicle(s), for example, a vector or vectors, expressing the recombinant antibodies (Abs) or antigen binding fragments thereof, or monomeric or dimeric antigen binding proteins as provided herein, including heavy and/or light chains, are episomal or are chromosomally integrated, for example, in a stable cell line capable of synthesizing, optionally inducible synthesizing, the recombinant antibodies (Abs) or antigen binding fragments thereof, or monomeric or dimeric antigen binding proteins as provided herein, or heavy and/or light chains as provided herein.

In alternative embodiments, provided are nucleic acids encoding recombinant antibodies (Abs) or antigen binding fragments thereof, or monomeric or dimeric antigen binding proteins as provided herein. Nucleic acids as provided herein can be made, isolated and/or manipulated by, for example, cloning and expression of cDNA libraries, amplification of message or genomic DNA by PCR, and the like. Nucleic acids used to practice embodiments as provided herein, whether RNA, cDNA, genomic DNA, vectors, viruses or hybrids thereof, may be isolated from a variety of sources, genetically engineered, amplified, and/or expressed/ generated recombinantly. Recombinant polypeptides generated from these nucleic acids can be individually isolated or cloned and tested for a desired activity. Any recombinant expression system can be used, including bacterial, fungal, mammalian, yeast, insect or plant cell expression systems, or hybrid or synthetic expression systems.

Alternatively, these nucleic acids can be synthesized in vitro by well-known chemical synthesis techniques, as described in, for example, Martin et al, ACS Synth. Biol. (2017) 6, 7, 1370-1379; Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, for example, subcloning, labeling probes (for example, random-primer labeling using Klenow polymerase, nick translation, amplification), sequencing, hybridization and the like are well described in the scientific and patent literature, see, for example, Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice embodiments as provided herein comprises screening and re-cloning inserts isolated or amplified from, for example, genomic clones or cDNA clones. Sources of nucleic acids include recombinant nucleic acid sequences, genomic or cDNA libraries contained and/or expressed in, for example, mammalian artificial chromosomes (MACs), see, for example, U.S. Pat. Nos. 5,721,118; 6,025,155; human artificial chromosomes, see, for example, Rosenfeld (1997) Nat. Genet. 15:333-335; yeast artificial chromosomes (YAC); bacterial artificial chromosomes (BAC); P1 artificial chromosomes, see, for example, Woon (1998) Genomics 50:306-316; P1-derived vectors (PACs), see, for example, Kern (1997) Biotechniques 23:120-124; cosmids, recombinant viruses, phages or plasmids.

In alternative embodiments, nucleic acids as provided herein are operably linked to transcriptional regulatory elements, including promoters, with can be constitutive or inducible transcriptional regulatory elements.

In alternative aspects, provided are “expression cassettes” comprising a nucleotide sequence as provided herein, for example encoding a recombinant antibody as provided herein. Expression cassettes can include at least a transcriptional regulatory element, for example, a promoter, operably linked with an antibody coding sequence, and optionally can also include transcription termination signals. Additional factors necessary or helpful in effecting expression may also be used, for example, enhancers.

In alternative aspects, expression cassettes used to practice embodiments as provided herein include plasmids, expression vectors, recombinant viruses, any form of recombinant “naked DNA” vector, and the like. In alternative aspects, an expression vehicle or a “vector” used to practice embodiments as provided herein can comprise a nucleic acid that can infect, transfect, transiently or permanently transduce a cell. In alternative aspects, a vector used to practice embodiments as provided herein can be a naked nucleic acid, or a nucleic acid complexed with protein or lipid. In alternative aspects, vectors used to practice embodiments as provided herein can comprise viral or bacterial nucleic acids and/or proteins, and/or membranes (for example, a cell membrane, a viral lipid envelope, etc.). In alternative aspects, vectors used to practice embodiments as provided herein can include, but are not limited to replicons (for example, RNA replicons, bacteriophages) to which fragments of DNA may be attached and become replicated. Vectors thus include, but are not limited to RNA, autonomous self-replicating circular or linear DNA or RNA (for example, plasmids, viruses, and the like, see, for example, U.S. Pat. No. 5,217,879), and can include both the expression and non-expression plasmids. In alternative aspects, the vector used to practice embodiments as provided herein can be stably replicated by the cells during mitosis as an autonomous structure, or can be incorporated within the host’s genome.

In alternative aspects, “promoters” used to practice embodiments as provided herein include all sequences capable of driving transcription of a coding sequence in a cell, for example, a bacterial, yeast, fungal, plant, insect (for example, baculovirus) or mammalian cell. Thus, promoters used in the constructs include cis-acting transcriptional control elements and regulatory sequences that are involved in regulating or modulating the timing and/or rate of transcription of a gene. For example, a promoter used to practice embodiments as provided herein can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences can interact with proteins or other biomolecules to carry out (turn on/off, regulate, modulate, etc.) transcription.

“Constitutive” promoters used to practice embodiments as provided herein can be those that drive expression continuously under most environmental conditions and states of development or cell differentiation. “Inducible” or “regulatable” promoters used to practice embodiments as provided herein can direct expression of a nucleic acid as provided herein under the influence of environmental conditions or developmental conditions. Examples of environmental conditions that may affect transcription by inducible promoters used to practice embodiments as provided herein include the presence of an inducing factor administered to a cell.

In alternative embodiments, polypeptides, including recombinant antibodies (Abs) or antigen binding fragments thereof, or monomeric or dimeric antigen binding proteins as provided herein or as used to practice methods or other embodiments as provided herein can comprise any “mimetic” and/or “peptidomimetic” form. In alternative embodiments, peptides and polypeptides used to practice embodiments as provided herein can comprise synthetic chemical compounds which have substantially the same structural and/or functional characteristics of the natural polypeptide, for example, a recombinant antibody as provided herein. The mimetic used to practice embodiments as provided herein can be either entirely composed of synthetic, non-natural analogues of amino acids, or, is a chimeric molecule of partly natural peptide amino acids and partly non-natural analogs of amino acids. The mimetic can also incorporate any amount of natural amino acid substitutions, for example, conservative amino acid substitutions, as long as such substitutions also do not substantially alter the mimetic’s structure and/or activity. Routine experimentation will determine whether a mimetic is effective for practicing embodiments as provided herein, for example, if a mimetic composition is effective in specifically binding a human p40 protein. Methodologies detailed herein and others known to persons skilled in the art may be used to select or guide one to choose effective mimetic for practicing the compositions and/or methods as provided herein.

Polypeptide mimetic compositions for practicing embodiments as provided herein can comprise any combination of non-natural structural components. In alternative aspects, mimetic compositions for practicing embodiments as provided herein can comprise one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, for example, a beta turn, gamma turn, beta sheet, alpha helix conformation, and the like. For example, a polypeptide can be characterized as a mimetic when all or some of its residues are joined by chemical means other than natural peptide bonds.

In alternative embodiments, an exemplary heavy chain variable region and/or light claim region comprises an amino acid sequence based on a sequence as set forth in SEQ ID NO:2 or SEQ ID NO:3, respectively, but having one or a plurality of amino acid residue deletions or substitutions, wherein optionally all or some of the amino acid substitutions are conservative amino acid substitutions, and wherein the amino acid sequence (or, the variant polypeptide) with the one or several deletions or substitutions at least substantially retains the ability to specifically bind to a peptide or epitope comprising SEQ ID NO:1, or a fragment of a polypeptide of SEQ ID NO:1, a p40 (or p63 isoform DeltaNp63) polypeptide or peptide or a fragment of a p40 polypeptide or peptide, albeit the specific binding of the variant can have a binding affinity higher or lower than the polypeptide of SEQ ID NO:2 or SEQ ID NO:3. In alternative embodiments, the variant polypeptide has between one and about 50 amino acid residue deletions or substitutions, wherein optionally all or some of the amino acid substitutions are conservative amino acid substitutions. In alternative embodiments, the variant polypeptide has between about 1 to 5, 5 to 10, 10 to 15 or 15 to 20 amino acid residue deletions or substitutions.

In alternative embodiments, an exemplary heavy chain variable region comprises an amino acid sequence as set forth in SEQ ID NO:2 having one, two, three, four, five, six, seven, eight, nine or ten, or between about 1 and 50, amino acid substitutions or deletions, wherein optionally all or some of the substitutions are conservative amino acid substitutions, and retaining the ability to substantially specifically bind to a peptide or epitope comprising SEQ ID NO:1, or a fragment of a polypeptide of SEQ ID NO:1, a p40 (or p63 isoform DeltaNp63) polypeptide or peptide or a fragment of a p40 polypeptide or peptide.

In alternative embodiments, an exemplary light chain variable region comprises an amino acid sequence as set forth in SEQ ID NO:3 having one, two, three, four, five, six, seven, eight, nine or ten, or between about 1 and 50, amino acid substitutions or deletions, wherein optionally all or some of the substitutions are conservative amino acid substitutions, and retaining the ability to substantially specifically bind to a peptide or epitope comprising SEQ ID NO:1, or a fragment of a polypeptide of SEQ ID NO:1, a p40 (or p63 isoform DeltaNp63) polypeptide or peptide or a fragment of a p40 polypeptide or peptide.

Conservative amino acid substitutions are those that substitute a given amino acid in a polypeptide by another amino acid of like characteristics. In alternative embodiments conservative substitutions are the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue. In alternative embodiments other variants are those in which one or more of the amino acid residues of a polypeptide of the invention includes a substituent group. Non-limiting examples of amino acids which may be substituted for an original amino acid in a protein and which may be regarded as conservative substitutions if there is little to no impact on the activity of the polypeptide include: Ala substituted with ser or thr; arg substituted with gln, his, or lys; asn substituted with glu, gln, lys, his, asp; asp substituted with asn, glu, or gln; cys substituted with ser or ala; gln substituted with asn, glu, lys, his, asp, or arg; glu substituted with asn, gln lys, or asp; gly substituted with pro; his substituted with asn, lys, gln, arg, tyr; ile substituted with leu, met, val, phe; leu substituted with ile, met, val, phe; lys substituted with asn, glu, gln, his, arg; met substituted with ile, leu, val, phe; phe substituted with trp, tyr, met, ile, or leu; ser substituted with thr, ala; thr substituted with ser or ala; trp substituted with phe, tyr; tyr substituted with his, phe, or trp; and val substituted with met, ile, leu.

### Purification and Isolation of Recombinant Proteins

In alternative embodiments, chimeric or the recombinant antibodies, antigen binding fragments thereof, or monomeric or dimeric antigen binding proteins, are substantially purified or isolated, and optionally the substantially purified or isolated forms are the forms used in immunohistochemistry (IHC) methodologies and/or as reagents, kits and/or products of manufacture as provided herein.

In alternative embodiments, chimeric or the recombinant antibodies, antigen binding fragments thereof, or monomeric or dimeric antigen binding proteins, are substantially purified or isolated using: physicochemical fractionation, for example, using differential precipitation, size-exclusion or solid-phase binding of immunoglobulins based on size, charge or other shared chemical characteristics of antibodies in typical samples; class-specific affinity, for example, solid-phase binding of particular antibody classes (for example, IgG or IgM) by immobilized biological ligands (for example, proteins, lectins, and the like) that have specific affinity to immunoglobulins, and this can purify all antibodies of the target class without regard to antigen specificity; or antigen-specific affinity, for example, affinity purification of only those antibodies in a sample that bind to a particular antigen molecule through their specific antigen-binding domains, where this purifies all antibodies that bind the antigen without regard to antibody class or isotype.

In alternative embodiments, chimeric or the recombinant antibodies, antigen binding fragments thereof, or monomeric or dimeric antigen binding proteins, are substantially purified or isolated using standard isolation methodologies such as chromatography, for example, Ion Exchange (IEX) Chromatography, Hydrophobic Interaction Chromatography (HIC), countercurrent chromatography, immunoaffinity and/or size exclusion chromatography.

In alternative embodiments, chimeric or the recombinant antibodies, antigen binding fragments thereof, or monomeric or dimeric antigen binding proteins, are generated in bioreactors, e.g., a perfusion bioreactor, using continuous expression and purification processes, e.g., as described by Vogg et al Methods Mol Biol. 2018; vol 1850:147-178, or using stirred-tank or rocking bioreactor systems, followed by purification.

### Immunohistochemistry

In alternative embodiments, immunohistochemistry methodologies and/or reagents used with the compositions (for example, a recombinant antibody (Ab), or antigen binding fragment thereof, or monomeric or dimeric antigen binding protein as provided herein capable of specifically binding a human p40 (p63 isoform DeltaNp63, or ΔNp63) protein or a polypeptide, or an antigen or an epitope comprising an amino acid sequence SEQ ID NO:1), products of manufacture, kits or methods as provided herein can include or comprise or comprise use of any IHC protocol, IHC armamentarium, device and/or image or data analysis system, for practicing IHC or IHC reagents known in the art, for example, as described in U.S. Pat. Nos. (USPNs) 10,634,590 (describing a slide holder assembly fixture for use in IHC); 10,565,479 (describing methods for identifying blurred areas in digital images of stained tissue); 10,564,076 (describing systems for analytical (or IHC) sample preparation); 10,551,395 (describing an automated histological staining system); 10,551,378 (describing a tissue staining method); 10,504,224 (describing a digital tissue image analysis system for IHC); 10,501,777 (describing simultaneous, multiplexed detection and quantification of protein expression in IHC); 10,488,340 (describing method for extracting an image of a target fluorophore in a biological material); 10,453,195 (describing methods of detecting tissue areas of interest using digital pathology imaging); 10,438,381 (describing devices, systems and methods for generating a digital image of a tissue section); 10,430,943 (describing methods and programs for automated nuclei area/number estimation for IHC image analysis); 10,416,176 (describing methods for processing specimens in an automated histological staining system); 10,393,633 (describing methods for processing and inhibiting the degradation of an IHC sample); 10,217,011 (describing handling of IHC slides); 10,209,165 (describing automated or semi-automated methods for assessing the quality of staining of a specimen containing cells); 10,126,216 (describing methods for fixing tissue samples for IHC); 9,423,322; 8,515,683 (describing methods and systems for automated detection of immunohistochemical (IHC) patterns), or U.S. Pat. application publication No. US 2019/0178867 A1 (describing detection of specific tissue objects within thin sections of tissue samples as imaged in a brightfield microscope without using a chromogenic stain that is specific to those tissue objects); US 2019/0156510 A1 (describing an image analysis method for analyzing an IHC tissue sample); or, US 2019/0080450 A1 (describing an automated determination of the staining quality of an IHC stained biological sample); or, US 2020/0316589 A1 (describing a multi-well solid support vessel for the processing and testing of fixed biological materials)..

In alternative embodiments, the recombinant antibodies, antigen binding fragments thereof, or monomeric or dimeric antigen binding proteins, in IHC protocols, or kits, as provided herein are substantially purified or isolated or are in the form of an unpurified or partially purified culture supernatant.

In alternative embodiments, methods as provided herein can use or comprise reagents for detecting or visualizing an antibody-antigen interaction using any products or methods know in the art, for example, and IHC protocol or reagents.

In alternative embodiments, methods as provided herein comprise use of chromogenic immunohistochemistry (CIH), wherein a primary antibody (for example, a recombinant antibody (Ab), or antigen binding fragment thereof, or monomeric or dimeric antigen binding protein, as provided herein) or secondary antibody (for example, where the secondary antibody binds to the primary antibody, or the recombinant antibody (Ab), or antigen binding fragment thereof, or monomeric or dimeric antigen binding protein as provided herein,) is conjugated to an enzyme such as peroxidase, for example, an immunoperoxidase), for example, a horseradish peroxidase (HRP), that can catalyze a color-producing reaction. In alternative embodiments, a chromogenic moiety used in methods as provided herein is or comprises a coumarin; a rhodamine; 2,3,6,7-tetrahydro-11-oxo-1H,5H,11H-[1]benzopyrano[6,7,8-ij]quinolizine-1- 0-carboxylic acid; 7-(diethylamino)coumarin-3-carboxylic acid; a coumarin derivative; a rhodamine derivative; a tetramethylrhodamine; a diarylrhodamine derivative; QSY 7; QSY 9; QSY 21; diazo chromophores; DABSYL; tartrazine; triarylmethane compounds; fast red; fast blue; fuchsin; Cascade Blue acetyl; Dapoxylsulfonic acid/carboxylic acid succinimidyl ester; DY-405; Alexa Fluor 405 succinimidyl ester; Cascade Yellow succinimidyl ester; pyridyloxazole succinimidyl ester (PyMPO); Pacific Blue succinimidyl ester; DY-415; 7-hydroxycoumarin-3-carboxylic acid succinimidyl ester; DYQ-425; 6-FAM phosphoramidite; Lucifer Yellow; iodoacetamide; Alexa Fluor 430 succinimidyl ester; Dabcyl succinimidyl ester; NBD chloride/fluoride; QSY 35 succinimidyl ester; DY-485XL; Cy2 succinimidyl ester; DY-490; Oregon Green 488 carboxylic acid succinimidyl ester; Alexa Fluor 488 succinimidyl ester; BODIPY 493/503 C3 succinimidyl ester; DY-480XL; BODIPY FL C3 succinimidyl ester; BODIPY FL C5 succinimidyl ester; BODIPY FL-X succinimidyl ester; DYQ-505; Oregon Green 514 carboxylic acid succinimidyl ester; DY-510XL; DY-481XL; 6-carboxy-4′,5′-dichloro-2′,7′-dimethoxyfluorescein succinimidyl ester (JOE); DY-520XL; DY-521XL; BODIPY R6G C3 succinimidyl ester; erythrosin isothiocyanate; 5-carboxy-2′,4′,5′,7′-tetrabromosulfonefluorescein succinimidyl ester; Alexa Fluor 532 succinimidyl ester; 6-carboxy-2′,4,4′,5′7,7′-hexachlorofluorescein succinimidyl ester (HEX); BODIPY 530/550 C3 succinimidyl ester; DY-530; BODIPY TMR-X succinimidyl ester; DY-555; DYQ-1; DY-556; Cy3 succinimidyl ester; DY-547; DY-549; DY-550; Alexa Fluor 555 succinimidyl ester; Alexa Fluor 546 succinimidyl ester; DY-548; BODIPY 558/568 C3 succinimidyl ester; Rhodamine red-X succinimidyl ester; QSY 7 succinimidyl ester; BODIPY 564/570 C3 succinimidyl ester; BODIPY 576/589 C3 succinimidyl ester; carboxy-X-rhodamine (ROX); succinimidyl ester; Alexa Fluor 568 succinimidyl ester; DY-590; BODIPY 581/591 C3 succinimidyl ester; DY-591; BODIPY TR-X succinimidyl ester; Alexa Fluor 594 succinimidyl ester; DY-594; carboxynaphthofluorescein succinimidyl ester; DY-605; DY-610; Alexa Fluor 610 succinimidyl ester; DY-615; BODIPY 630/650-X succinimidyl ester; erioglaucine; Alexa Fluor 633 succinimidyl ester; Alexa Fluor 635 succinimidyl ester; DY-634; DY-630; DY-631; DY-632; DY-633; DYQ-2; DY-636; BODIPY 650/665-X succinimidyl ester; DY-635; Cy5 succinimidyl ester; Alexa Fluor 647 succinimidyl ester; DY-647; DY-648; DY-650; DY-654; DY-652; DY-649; DY-651; DYQ-660; DYQ-661; Alexa Fluor 660 succinimidyl ester; Cy5.5 succinimidyl ester; DY-677; DY-675; DY-676; DY-678; Alexa Fluor 680 succinimidyl ester; DY-679; DY-680; DY-682; DY-681; DYQ-3; DYQ-700; Alexa Fluor 700 succinimidyl ester; DY-703; DY-701; DY-704; DY-700; DY-730; DY-731; DY-732; DY-734; DY-750; Cy7 succinimidyl ester; DY-749; DYQ-4; Cy7.5 succinimidyl ester; 7-diethylaminocoumarin-3-carboxylic acid; succinimidyl ester; Dabsyl sulfonyl chloride; fluorescein isothiocyanate (FITC) carboxy succinimidyl ester (DY-495); Rhodamine Green carboxylic acid succinimidyl ester (DY-505); eosin isothiocyanate (EITC); 6-carboxy-2′,4,7,7′-tetrachlorofluorescein succinimidyl ester (TET); carboxyrhodamine 6G succinimidyl ester; carboxytetramethylrhodamine succinimidyl ester (TMR, TAMRA) (DY-554); QSY 9 succinimidyl ester; sulforhodamine B sulfonyl chloride (DY-560); Texas Red (sulforhodamine 101); gallocyanine; Fast Green FCF; Malachite Green; or, a QSY 21 succinimidyl ester.

In alternative embodiments, methods as provided herein comprise use of immunofluorescence, where a primary or a secondary antibody is tagged to a fluorophore, such as fluorescein or fluorescein isothiocyanate (FITC), a triarylmethane dye such as rhodamine or rhodamine derivatives (for example, tetramethylrhodamine (TRITC), rhodamine 6G, rhodamine 123, rhodamine B, carboxytetramethylrhodamine (TAMRA), tetramethylrhodamine (TMR), sulforhodamine 101), aminomethylcoumarin acetate (AMCA), ALEXA™ or DYLIGHT™ fluors, or a fluorophore or dye as described in U.S. Pat. application No. US 2019/0018018 A1. 3,3′-Diaminobenzidine (DAB) also can be used.

In alternative embodiments, methods as provided herein comprise use of a direct method or one-step staining method where a primary antibody (for example, recombinant antibodies (Ab), or antigen binding fragments thereof, or monomeric or dimeric antigen binding proteins as provided herein) is labeled and reacts directly with an antigen, for example, in a tissue sections. While this technique utilizes only one antibody and therefore is simple and rapid, the sensitivity may be lower due to little signal amplification.

In alternative embodiments, methods as provided herein comprise use of an indirect method where an unlabeled primary antibody (first layer) binds to a target antigen (for example, p40), for example, in a tissue or organ, and a labeled secondary antibody (second layer) then is reacted with the primary antibody. The secondary antibody can be against the isotype, for example, IgG, of the animal species in which the primary antibody is derived. This method can be more sensitive than direct detection strategies because of signal amplification due to the binding of several secondary antibodies to each primary antibody if the secondary antibody is conjugated to a detecting agent such as a fluorescent or enzyme reporter.

In alternative embodiments, further amplification is achieved if the secondary antibody is conjugated to several detecting molecules, for example, biotin molecules, which can recruit complexes of avidin-, streptavidin- or NEUTRAVIDIN™ protein-bound enzyme.

In alternative embodiments, the IHC is performed on tissue sections or tissue biopsies, for example, paraformaldehyde (PFA) fixed tissues or organs, or formalin-fixed paraffin-embedded tissues. In alternative embodiments, a tissue is sectioned or sliced or used whole. Before sectioning, the tissue sample can be embedded in a medium, for example, paraffin wax or cryomedia. Tissue sections can be sectioned or sliced on a variety of instruments, most commonly using a microtome, cryostat, or vibratome. Specimens can be sectioned or sliced at a range of about 3 µm to 5 µm. The sections or slices can be mounted on slides, dehydrated using alcohol washes of increasing concentrations (for example, 50%, 75%, 90%, 95%, 100%), and cleared using a detergent like xylene before being imaged under a microscope.

Depending on the method of fixation and tissue preservation, the sample may require additional steps to make a p40 epitope available for antibody binding, including deparaffinization and antigen retrieval. For formalin-fixed paraffin-embedded tissues, antigen-retrieval is often necessary, and can comprise pre-treating the sections with heat or proteases.

In alternative embodiments, the IHC is performed using an ENVISION DUOFLEX DOUBLESTAIN SYSTEM™ (EnVision DuoFLEX Doublestain System) (Agilent, San Jose, CA), which allows for staining of two or more markers on a single slide. In alternative embodiments, the IHC is performed using an EnVision FLEX HRP Magenta, High pH (Dako Omnis) system, and binding can be visualized by EnVision FLEX HRP Magenta Chromogen. In alternative embodiments, the IHC is performed using En Vision FLEX Mini Kit, High pH, which is a high-sensitivity visualization system intended for use in IHC together with Dako AUTOSTAINER™ instruments; this dual link system detects primary mouse and rabbit antibodies and the reaction is visualized by 3,3′-Diaminobenzidine (DAB) chromogen (DAB forms a water-insoluble brown precipitate when oxidized, for example, by a peroxidase).

### Products of Manufacture and Kits

Provided are products of manufacture and kits comprising chimeric or recombinant anti-human p40 Abs as provided, and for practicing methods as provided herein using the chimeric or recombinant anti-human p40 Abs as provided herein; and optionally the products of manufacture and/or kits can further comprise some or all reagents needed to perform an immunohistochemistry (IHC), and optionally can comprise instructions for practicing methods as provided herein.

In alternative embodiment, products of manufacture have attached thereto or affixed (optionally covalently bound) on or onto a chimeric or recombinant antibody or a dimeric antigen binding protein as provided herein, and optionally products of manufacture as provided herein are or comprise arrays, chips, biochips, slides, trays, dishes (for example, microtiter dishes), phages or phagemids.

In alternative embodiment, immunohistochemistry methodologies and/or reagents used to practice composition, product of manufacture (for example, kit) or method embodiments as provided herein can include or comprise or comprise use of any IHC protocol, IHC armamentarium, device and/or image or data analysis system, for practicing IHC or IHC reagents known in the art, for example, as described in U.S. Pat. Nos. 10,565,479 (describing methods for identifying blurred areas in digital images of stained tissue); 10,564,076 (describing systems for analytical (or IHC) sample preparation); 10,551,395 (describing an automated histological staining system); 10,551,378 (describing a tissue staining method); 10,504,224 (describing a digital tissue image analysis system for IHC); 10,501,777 (describing simultaneous, multiplexed detection and quantification of protein expression in IHC); 10,488,340 (describing method for extracting an image of a target fluorophore in a biological material); 10,453,195 (describing methods of detecting tissue areas of interest using digital pathology imaging); 10,438,381 (describing devices, systems and methods for generating a digital image of a tissue section); 10,416,176 (describing methods for processing specimens in an automated histological staining system); 10,393,633 (describing methods for processing and inhibiting the degradation of an IHC sample); 10,217,011 (describing handling of IHC slides); 10,209,165 (describing automated or semi-automated methods for assessing the quality of staining of a specimen containing cells); 10,126,216 (describing methods for fixing tissue samples for IHC); 9,423,322.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 85%, 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR - Basics: From Background to Bench, First Edition, Springer Verlag, Germany.

### Example 1: Development of Exemplary Anti-p40 Antibody Clone 15F11

This example describes the development of the exemplary anti-p40 antibody clone designated “15F11”.

Peptide MLYLENNAQTQFSEPQC-NH2 (SEQ ID NO:1) was synthetized and used for immunization of 3 rabbits.

After 47 days blood samples were taken from the rabbits and the titer determined by ELISA titration against the peptide immunogen.

### ELISA Protocol

coating: overnight (o/n) 4° C. Plate 100 ng/well neutravidine MS544.9 in PBS, wash plates 3 times with PBST;
blocking: 4.5 hours (h) room temperature (RT) block each well with 370 µl 1% BSA in PBST;
coating: 1 h 37° C. 100 ng/well biotin-antigen (MS1891.2) in 1% BSA (bovine serum albumin) in PBST, wash plates 3 times with PBST;
samples: 1.5 h RT add plasma dilution series, as mentioned in plate format (see results), in 100 µl 1% BSA in PBST per well, wash plates 3 times with PBST;
detection antibody: 1.25 h RT add 100 µl Goat anti Rabbit IgG-HRP (MS169.4): 1/5000 in 1% BSA in PBST, wash plates 3 times with PBST. wash plates 3 times with PBS;
staining: add 50 µl TMB per well;
stop reaction: 7 minutes add 50 µl 2 M H₂SO₄ per well, measure A450.

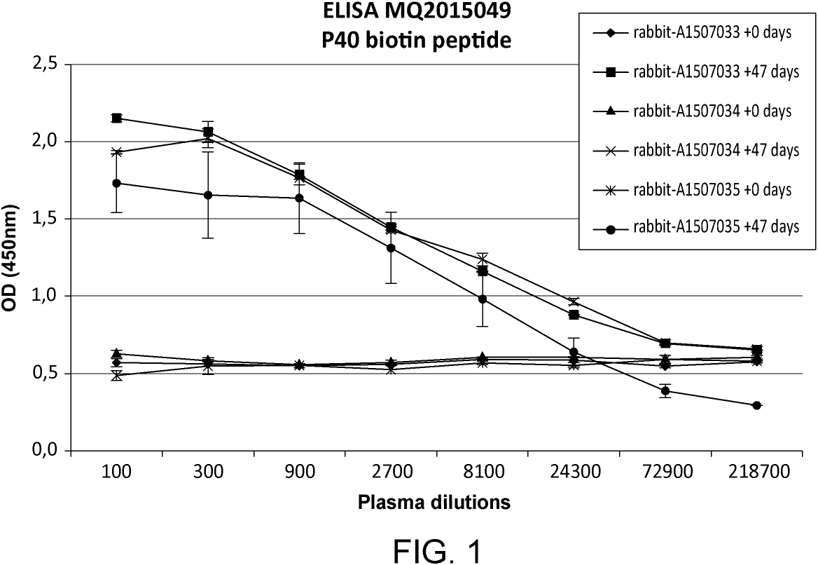

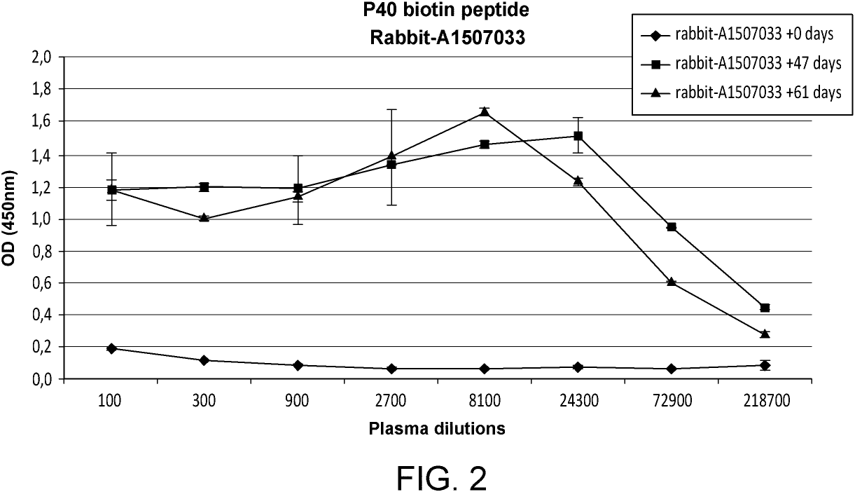

Results (optical density OD as a function of plasma dilutions) are graphically illustrated in FIG. 1.

It was decided to continue immunizations to achieve a higher titer. Retest was made at day 61 after immunization start, and ELISAs were repeated, and results are graphically illustrated in FIG. 2, FIG. 3 and FIG. 4. All rabbits had a higher plasma titer against p40 biotin peptide.

Next step: Select and culture target reactive B-cells and screen B-cell supernatants to identify the wells containing target reactive B-cells.

B-cell selection and culture was performed using spleen cells of one rabbit, followed by screening B-cell culture supernatants in ELISA on P40 biotin peptide.

Results: B-cell selection was performed, resulting in a successful selection of target reactive B-cells analyzed by target ELISA using B-cell culture supernatants. In both selection methods a subset of selected B-cells was cultured single cell per well and a subset as a back-up- multiple cells per well. Clones were selected based on measured OD450 great than (>) 0.2 in the antigen ELISA (5 times background) single cell per well and multiple cells per well, respectively.

B-cell culture supernatants positive in ELISA were further functionally tested in paraffin IHC (IHC-P) to identify clones producing antibody capable of binding the p40 antigen in FFPE (formalin fixed paraffin embedded) tissue.

The nucleic acids encoding the variable regions of the antibodies which specifically bind to p40 from the well with B cells exhibiting desirable results in the IHC were cloned and sequenced. Additionally, a small scale expression of full length recombinant rabbit IgG was made in eukaryotic cells and tested by standard IHC protocol.

### Cloning and Sequencing

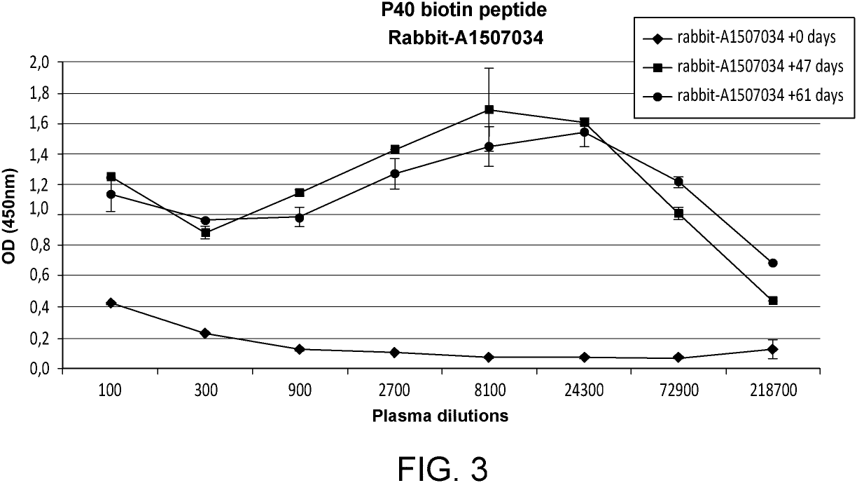

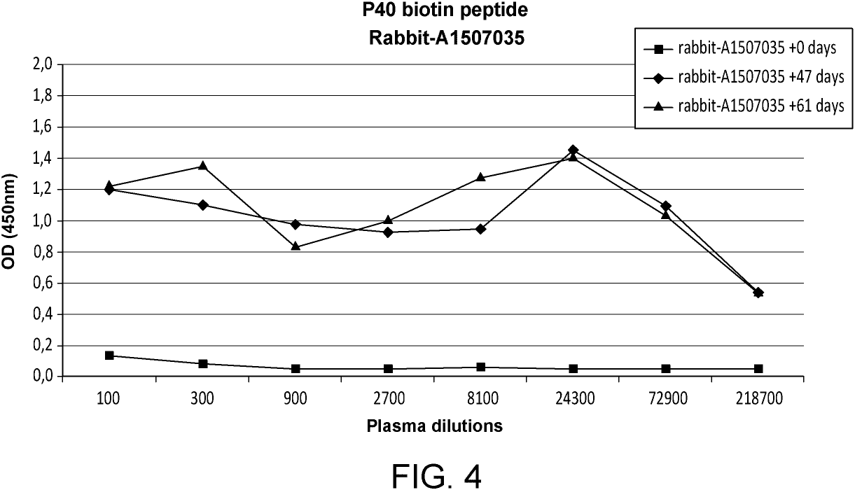

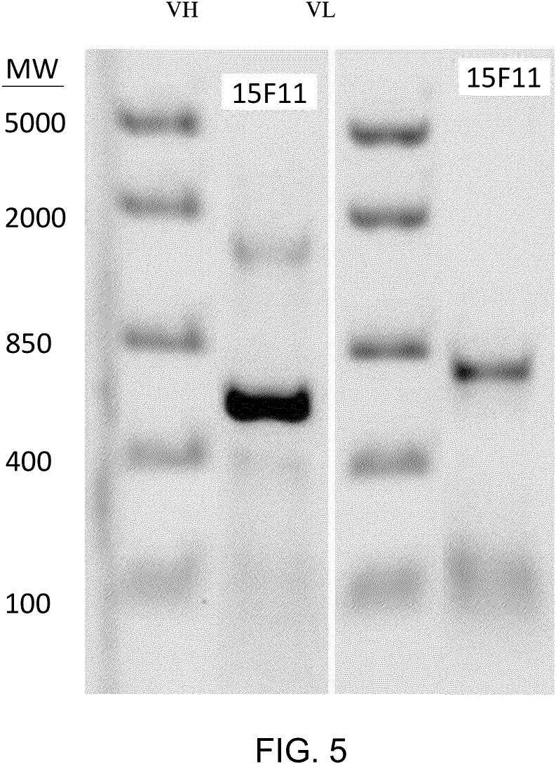

As illustrated in FIG. 5, PCR reactions on cDNA derived from B-cell cultures to amplify the heavy and light variable domains yielded PCR products of the correct size; FIG. 5 illustrates PCR-amplified VH and VL domains of the 4 selected clones.

Gel purified nucleic acids encoding VH and VL domains of all 4 clones were cloned into vectors, respectively, and subsequently subjected to colony PCR for sequence analysis. Sequences were translated and aligned.

### Expression and Validation

The sequences obtained from the respective VH and VL cloning were used to synthesize codon optimized gene constructs for the respective VH and VL fragments. These were subcloned into an expression vector (pTT5 backbone) containing the rabbit constant heavy and light chains, respectively. Per clone the obtained recombinant sequence were expressed in the mammalian (HEK293-6E) expression system, producing recombinant monoclonal rabbit anti-human p40 antibody.

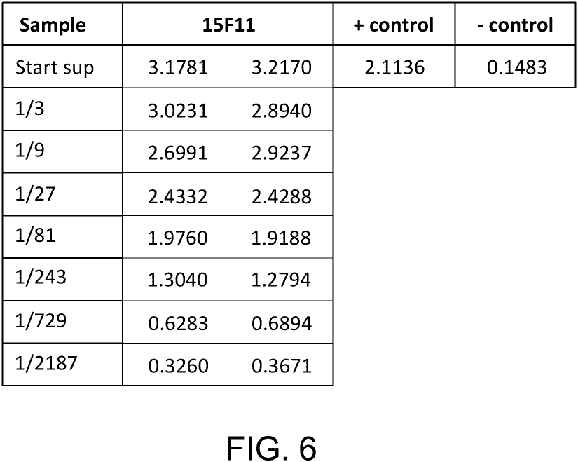

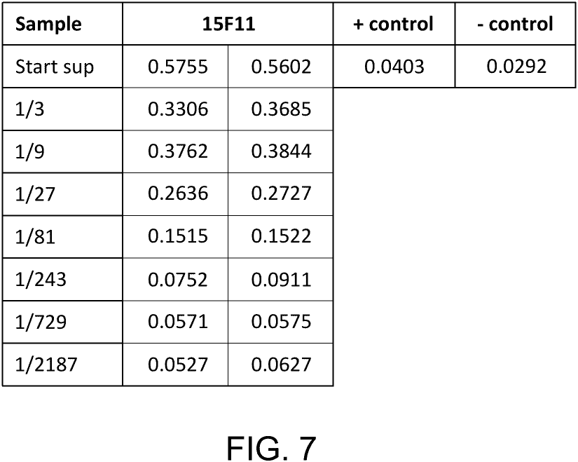

Target ELISA was performed in duplicate to validate immunoreactivity of the expressed antibodies on the antigen (Table 1, FIG. 6) and a biotinylated control peptide (Table 2, FIG. 7). Control plasma from rabbit A150733 (plus 47 days; 1:20,000 diluted) and PBST plus 1% BSA were included as positive and negative control, respectively. Reactivity screening revealed that all 4 re-cloned antibodies show strong reactivity against client’s target (Table 1, FIG. 6), while no/limited reactivity against an included irrelevant biotinylated peptide was observed (Table 2, FIG. 7).

Table 1 (as illustrated in FIG. 6): ELISA-based reactivity analysis of recloned rabbit B-cell clones derived from B-cell selection and culture against client’s target P40 biotin peptide (MS1891.2).

Table 2 (as illustrated in FIG. 7): ELISA-based reactivity analysis of recloned rabbit B-cell clones derived from B-cell selection and culture against an irrelevant biotinylated peptide.

### Conclusions:

Clone 15F11 was successfully cloned, sequenced, expressed and validated for specific reactivity against client’s target (MS1891.2) in ELISA.
Except for 1 amino acid difference in framework 4 of the VH, the variable domain sequences of 2 clones are identical.
Approximately 1.8 ml supernatant containing the corresponding rabbit IgG1 antibodies are used for further screening.

### Staining Performance of Clone 15F11:

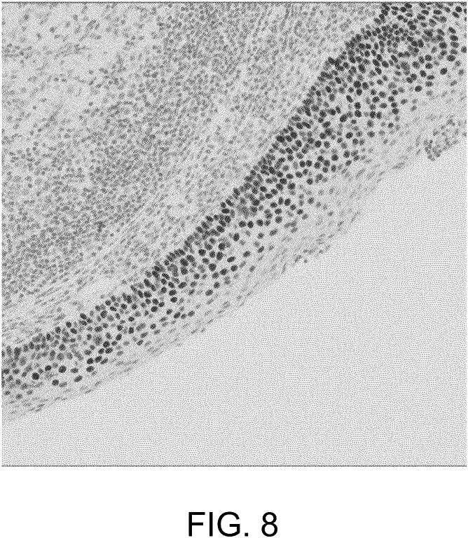

FIG. 8 illustrates an image of clone 15F11 antibodies staining squamous epithelial cells of tonsil.

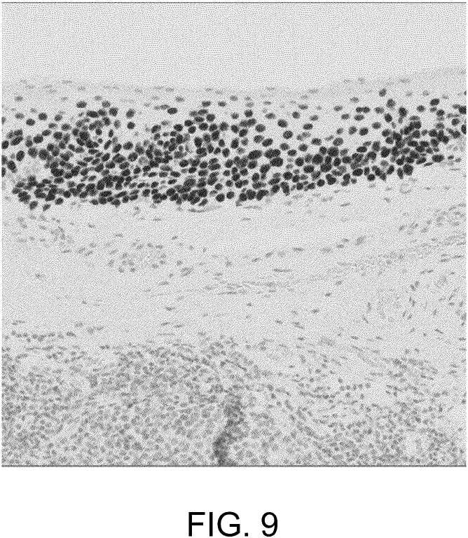

FIG. 9 illustrates an image of clone 15F11 antibodies staining squamous epithelial cells of tonsil.

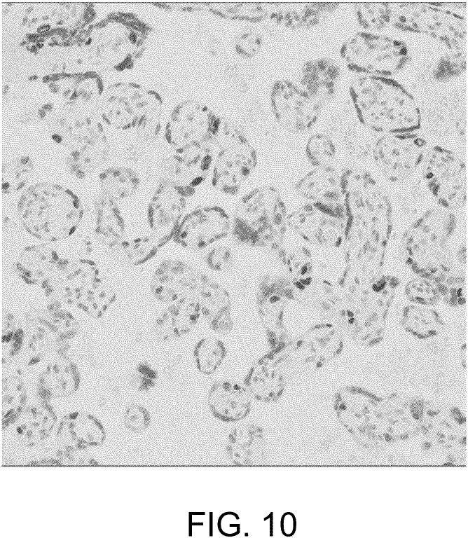

FIG. 10 illustrates an image of clone 15F11 antibodies staining cytotrophoblasts of placenta.

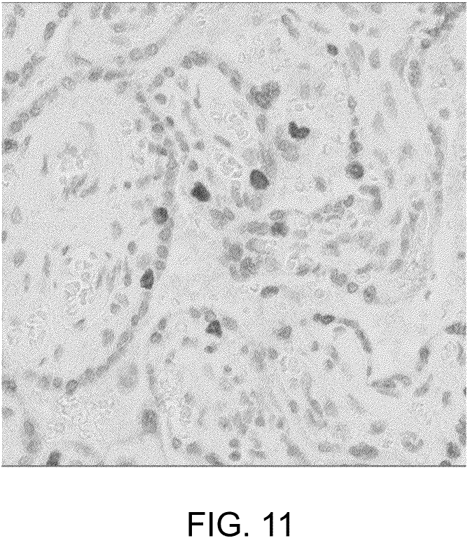

FIG. 11 illustrates an image of clone 15F11 antibodies staining cytotrophoblasts of placenta.

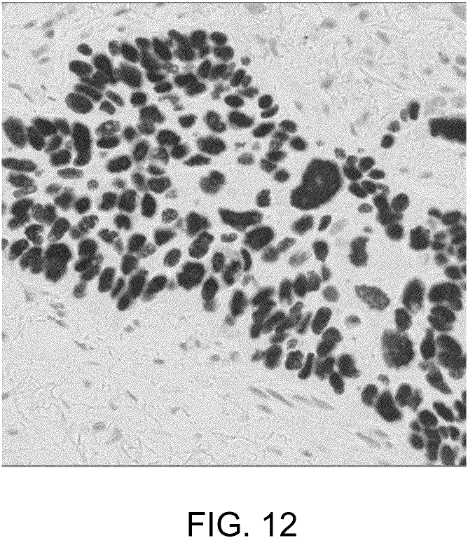

FIG. 12 illustrates an image of clone 15F11 antibodies staining lung squamous cell carcinoma.

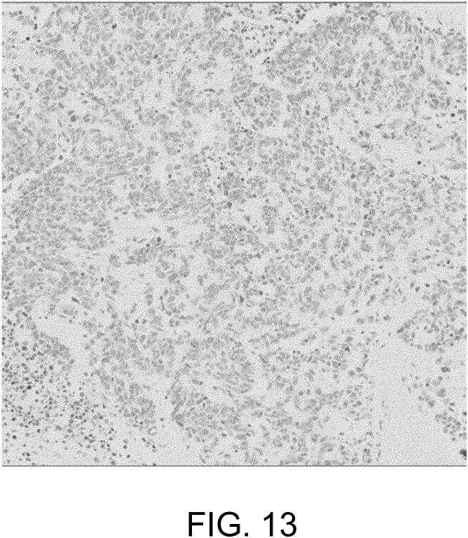

FIG. 13 illustrates an image of clone 15F11 antibodies staining lung adenocarcinoma.

### IHC on Cancer Cells

IHC was used to test the recombinant rabbit monoclonal anti-human p40 antibodies, an IgG isotypes, on a panel of cancer cells and tissues, as illustrated in the table of TABLE 3, where: -: negative, (+):≥ 1-9% positive cells, +: ≥ 10% positive cells.

### Results for IHC on Cancer Cells

### mAb Clone BC28:

mAb clone BC28, Biocare Medical, was used as reference for the two p40 antibodies tested. The protocol was performed on the Ventana BenchMark Ultra. A weak to strong nuclear staining reaction was seen in squamous epithelial cells of esophagus and tonsil, and basal cells of prostate glands. A weak to moderate staining reaction was observed in dispersed trophoblasts of placenta. No staining reaction was seen in endothelial cells, muscle cells and lymphocytes. In general, a high signal to noise ratio was provided.

The neoplasias staining profile is listed above. 21/23 lung squamous cell carcinomas were positive using a cut-off greater than or equal to (≥) 10%. All 23 tested lung adenocarcinomas were negative.

### rmAb Clone 15F11:

In this test, the best protocol with shortest TAT and highest signal-to-noise ratio was found by using a titer of 1:250 of the primary Ab, heat induced epitope retrieval (HIER) in Target Retrieval Solution™ (TRS) (Agilent, Dako) high pH and using EnVision FLEX+™ as detection system. If using HIER in TRS Low pH a titer of 1:50 gave the best result.

Virtually the result for the two selected protocols for rmAb clone 15F11 were identical. Both demonstrated a moderate to strong nuclear staining reaction in squamous epithelial cells of esophagus and tonsil, and basal cells of prostate glands. In dispersed trophoblasts of placenta, a weak to moderate nuclear staining reaction was seen. An increased titer for both protocols gave an inferior signal-to-noise ratio.

The staining pattern for the neoplasias were similar to the reference Ab.

### Conclusion for IHC on Cancer Cells

In this test performed, the best overall result was obtained by the recombinant (rmAb) clone 15F11 with HIER in TRS High pH. The result was comparable to the reference based on mAb BC28 (used as a control) stained on BenchMark Ultra™, Ventana.

### IHC Tissue Staining

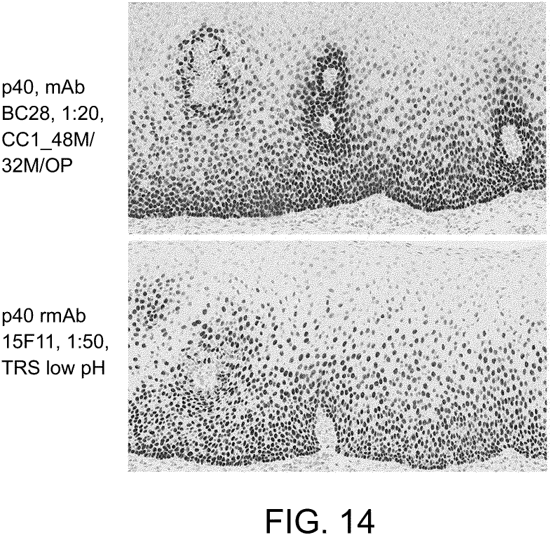

FIG. 14 illustrates an image of anti-p40 clone 15F11, and BC28 antibodies staining esophagus (100X image).

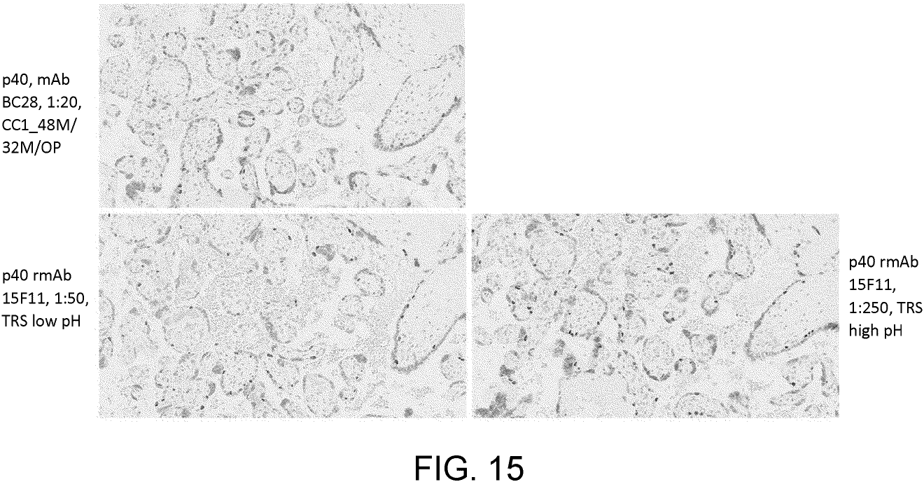

FIG. 15 illustrates an image of anti-p40 clone 15F11, and BC28 antibodies staining placenta (100X image) under various conditions, as indicated in the figure.

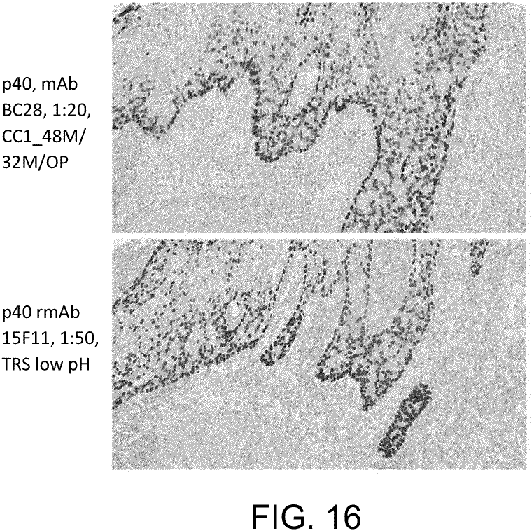

FIG. 16 illustrates an image of anti-p40 clone 15F11, and BC28 antibodies staining tonsil (24 hour fixation, 100X image).

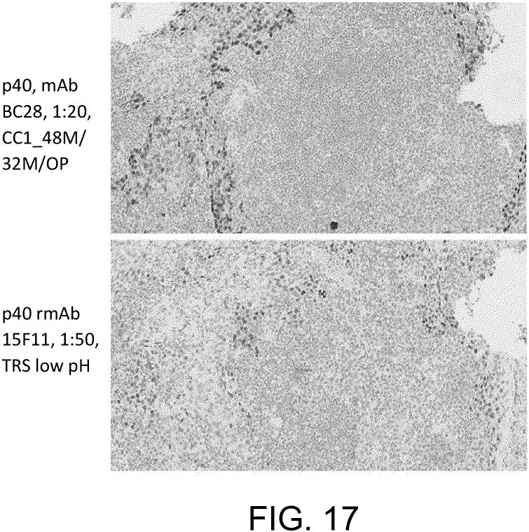

FIG. 17 illustrates an image of anti-p40 clone 15F11, and BC28 antibodies staining tonsil (144 hour fixation, 100X image).

FIG. 18 illustrates an image of anti-p40 clone 15F11, and BC28 antibodies staining normal prostate (100X image) under various conditions, as indicated in the figure.

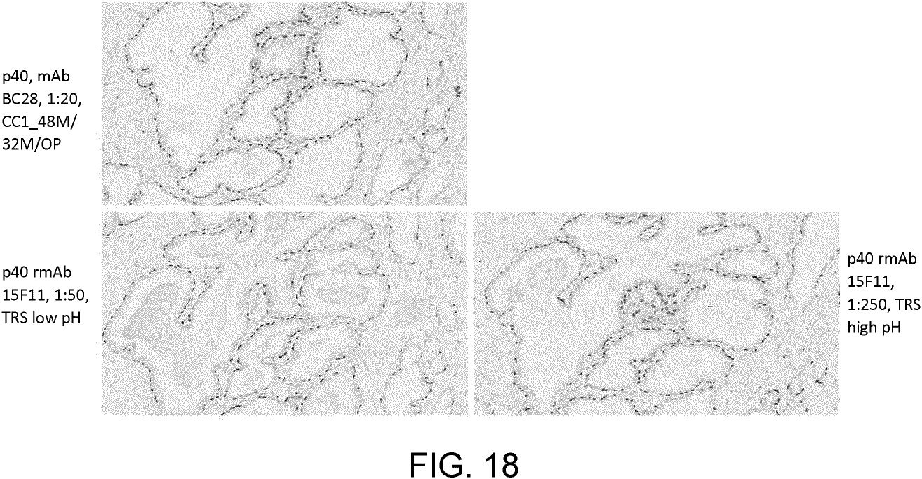

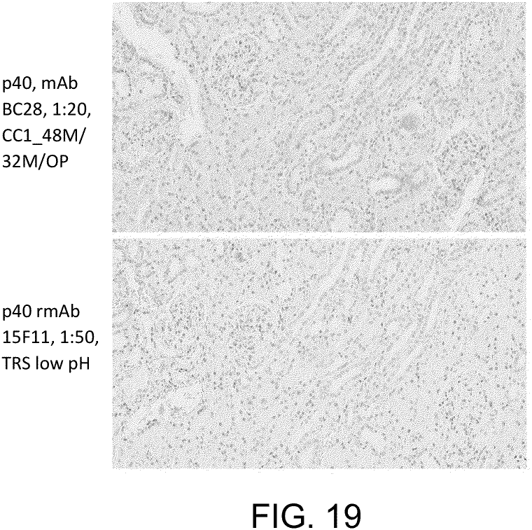

FIG. 19 illustrates an image of anti-p40 clone 15F11, BC28 antibodies staining kidney (100X image).

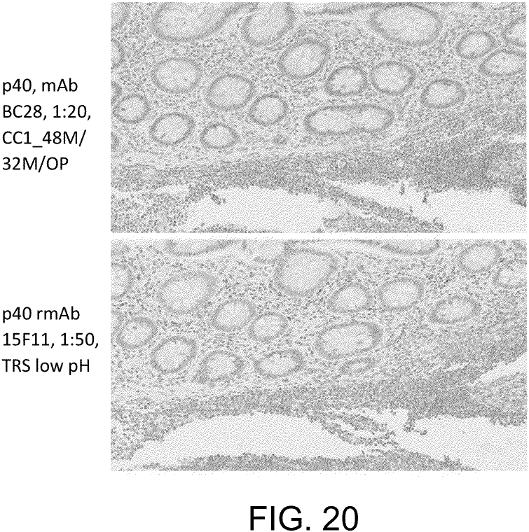

FIG. 20 illustrates an image of anti-p40 clone 15F11, BC28 antibodies staining appendix (100X image).

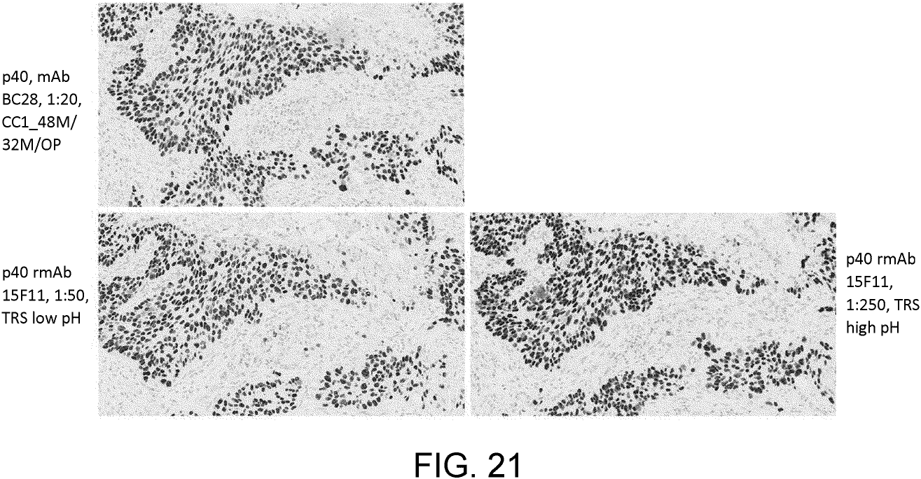

FIG. 21 illustrates a first image of anti-p40 clone 15F11, BC28 antibodies staining lung squamous cell carcinoma (100X image) under various conditions, as indicated in the figure.

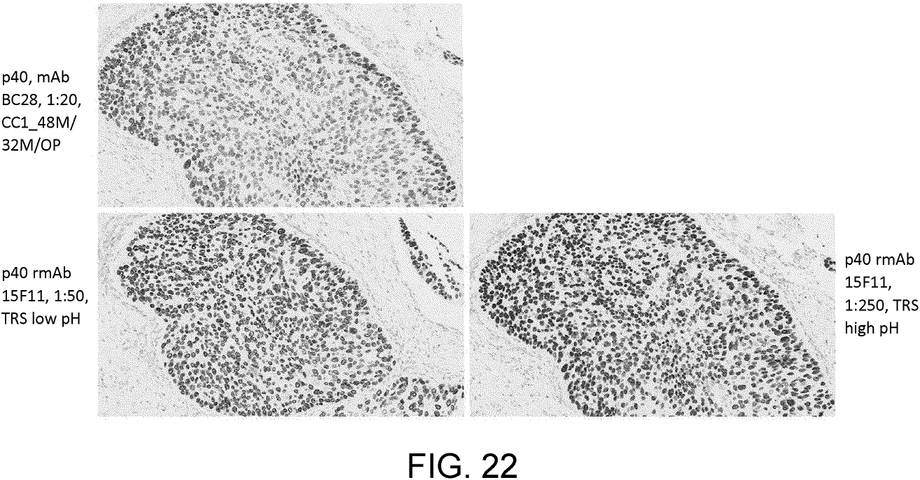

FIG. 22 illustrates a second image of anti-p40 clone 15F11, BC28 antibodies staining lung squamous cell carcinoma (100X image) under various conditions, as indicated in the figure.

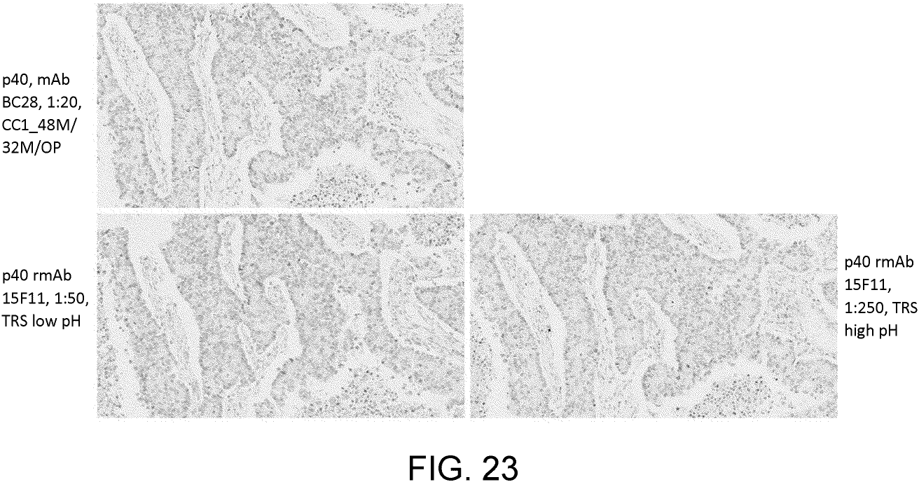

FIG. 23 illustrates a first image of anti-p40 clone 15F11, BC28 antibodies staining lung adenocarcinoma (100X image) under various conditions, as indicated in the figure.

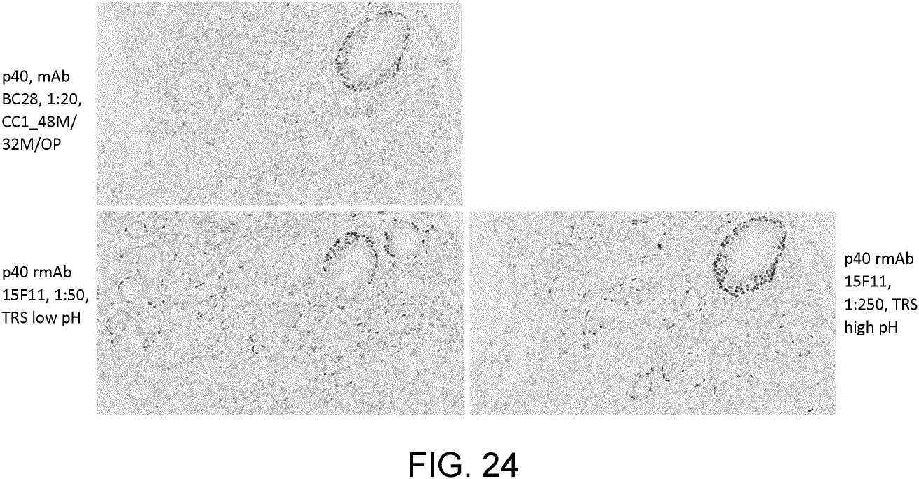

FIG. 24 illustrates a second image of anti-p40 clone 15F11, BC28 antibodies staining lung adenocarcinoma (100X image) under various conditions, as indicated in the figure.

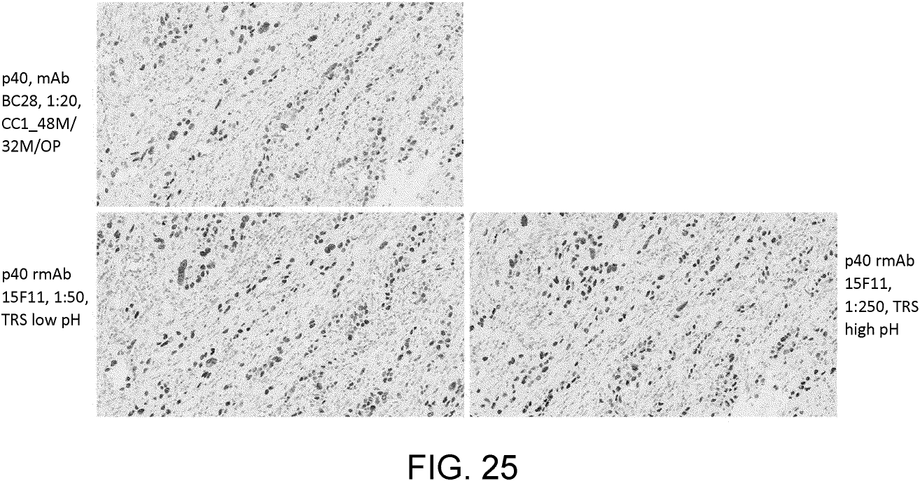

FIG. 25 illustrates an image of anti-p40 clone 15F11, BC28 antibodies staining lung large cell carcinoma (100X image) under various conditions, as indicated in the figure.

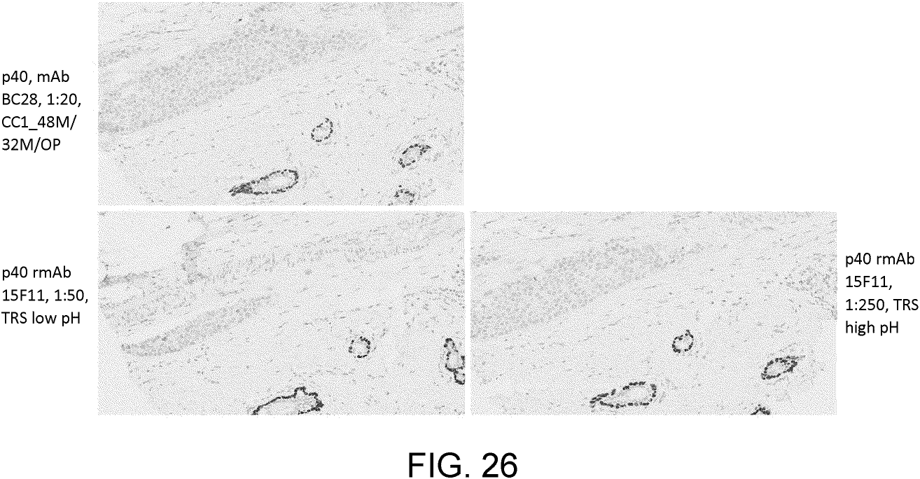

FIG. 26 illustrates an image of anti-p40 clone 15F11, BC28 antibodies staining mamma ductal carcinoma (100X image) under various conditions, as indicated in the figure.

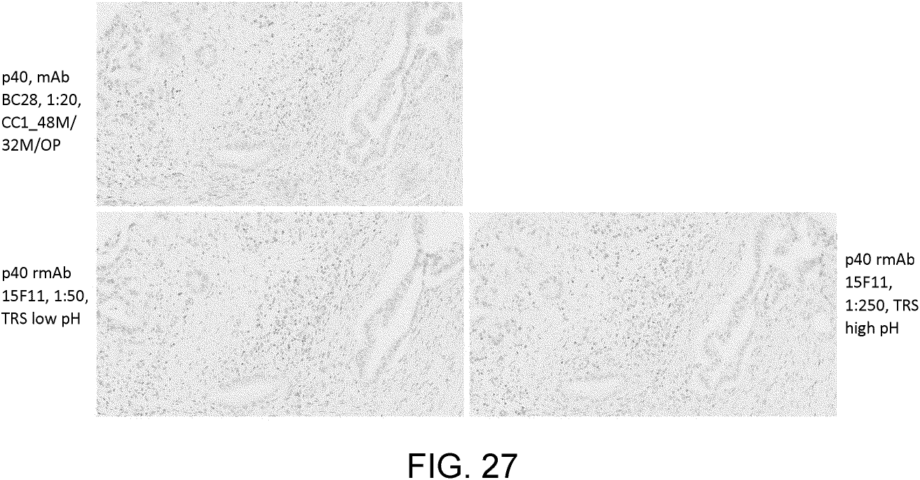

FIG. 27 illustrates an image of anti-p40 clone 15F11, BC28 antibodies staining pancreatic adenocarcinoma (100X image) under various conditions, as indicated in the figure.

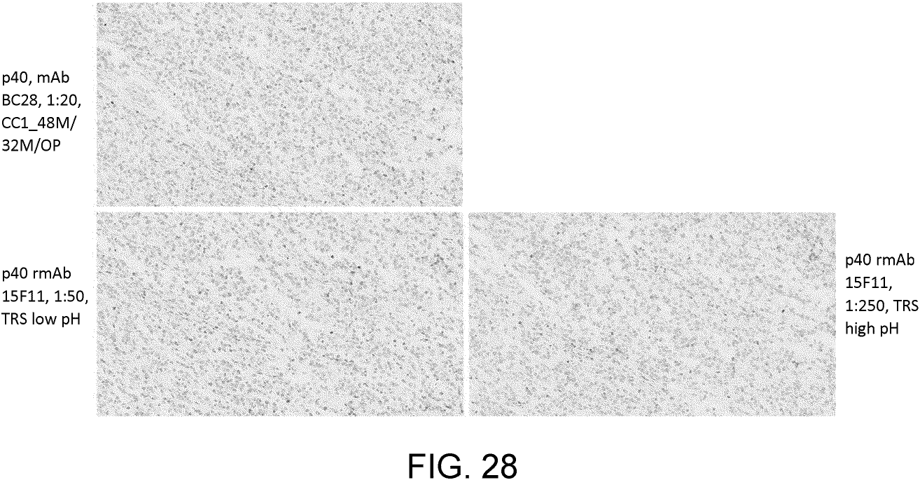

FIG. 28 illustrates an image of anti-p40 clone 15F11, BC28 antibodies staining diffuse large B-cell lymphoma (DLBCL) (100X image) under various conditions, as indicated in the figure.

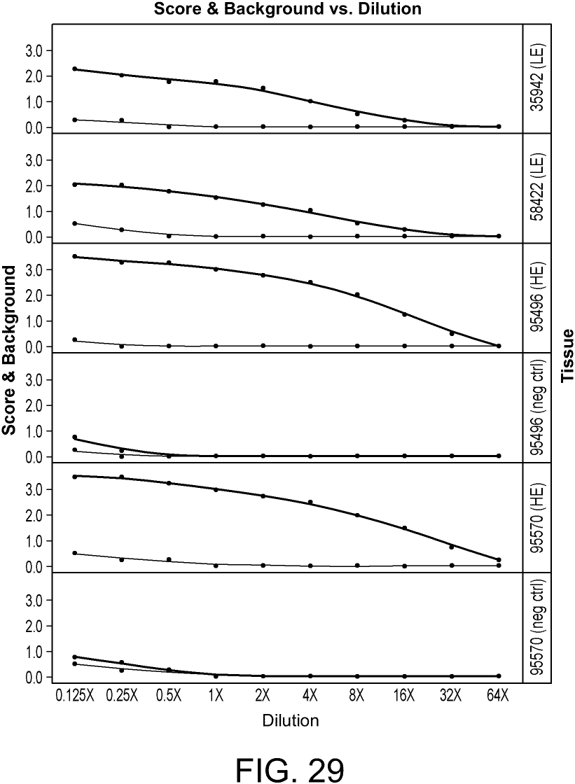

FIG. 29 Positive normal tonsil (p40 High Expressing (HE) and p40 Low Expression (LE)) and negative control tissue tested with anti-p40 clone 15F11. IHC intensity score (blue line) and background staining (red line) vs. antibody dilution. The normal tonsil tissues #95496 and #95570 were evaluated for both HE structures (squamous epithelial cells of the tonsil) and negative control structures (non-epithelial cells of the tonsil).

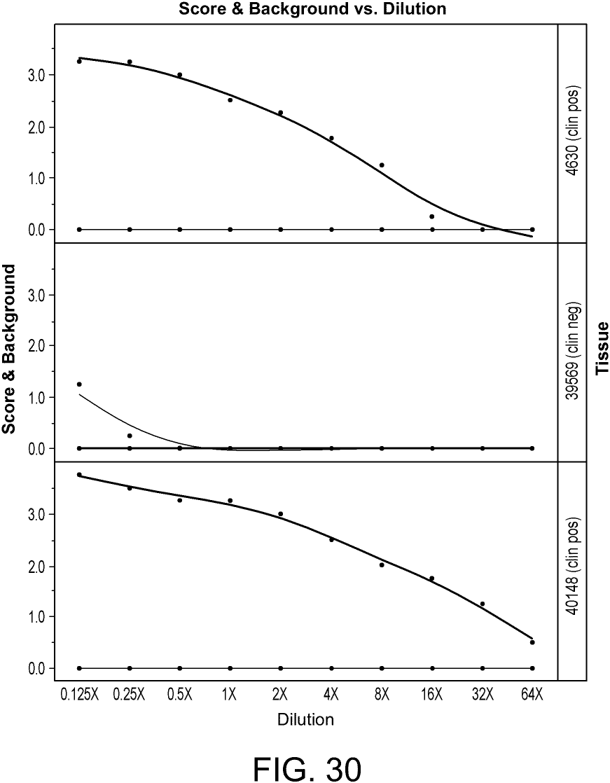

FIG. 30 Lung squamous cell carcinoma (clin pos) and negative lung adenocarcinoma (clin neg) tissues were tested with two-fold dilutions of the antibody. Specific staining (blue line) and background staining (red line) vs. the antibody dilution. 2X represents half the 1X concentration etc.

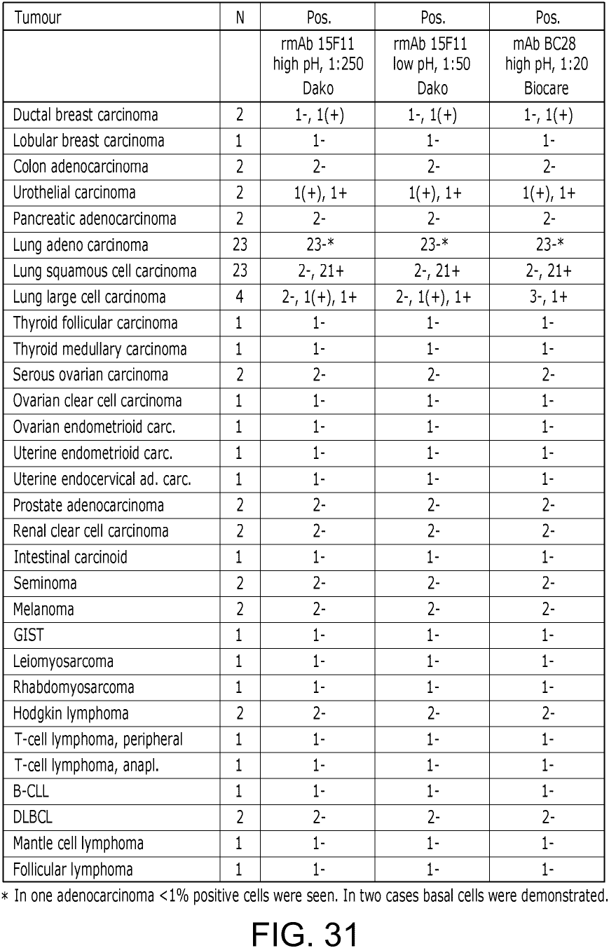

FIG. 31 illustrates TABLE 3, showing data from IHC used to test the exemplary recombinant rabbit monoclonal anti-human p40 antibodies on a panel of cancer cells and tissues.

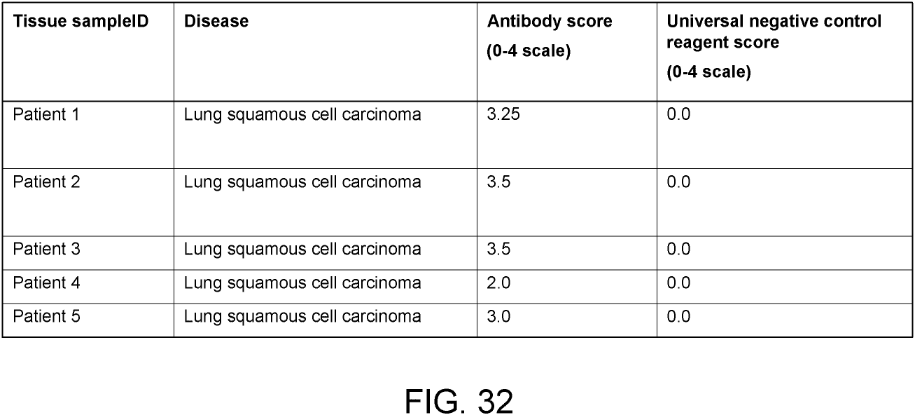

FIG. 32 illustrates TABLE 4, showing data from staining with anti-p40 clone 15F11 as well as a negative control reagent.

### REFERENCES

1. Bishop, J.A., et al., p40 (DeltaNp63) is superior to p63 for the diagnosis of pulmonary squamous cell carcinoma. Mod Pathol, 2012. 25(3): p. 405-15.

2. Sailer, V., et al., Comparison of p40 (DeltaNp63) and p63 expression in prostate tissues--which one is the superior diagnostic marker for basal cells? Histopathology, 2013. 63(1): p. 50-6.

A number of embodiments of the invention have been described. Nevertheless, it can be understood that various modifications may be made without departing from the spirit and scope of the invention. Accordingly, other embodiments are within the scope of the following claims.

What is claimed is:

1. A recombinant antibody (Ab), or antigen binding fragment thereof, or monomeric or dimeric antigen binding protein, capable of specifically binding a human p40 (p63 isoform DeltaNp63, or ΔNp63) protein or a polypeptide, or an antigen or an epitope comprising an amino acid sequence MLYLENNAQTQFSEPQC-NH2 (SEQ ID NO:1).

2. The recombinant antibody (Ab), or antigen binding fragment thereof, or monomeric or dimeric antigen binding protein, of claim 1, fabricated as or in the form of:
an antigen-binding fragment (Fab, or an Ab fragment having just one constant and one variable domain of each of an Ab heavy and light chain),
a F(ab′)₂ (or an Ab digested by pepsin yielding two fragments: a F(ab′)₂ fragment and a pFc′ (pepsin cleavage Fc) fragment),
a Fab′ (a single chain of a F(ab′)₂ fragment),
a single-chain variable fragment (scFv) (or a fusion protein of a variable region of an Ab heavy and light chain connected together with a linker peptide optionally of about ten to about 25 amino acids in length),
a (scFv)₂, or a di-scFv or a bi-scFv, or a single peptide chain having two variable heavy and two variable light regions yielding tandem scFv,
a minibody (or a fusion protein of a variable region of an Ab heavy and light chain connected together with an alkyl group, optionally a methyl or an ethyl group)
a diabody (or an scFv with a linker peptide too short (optionally about five amino acids) for the two variable regions to fold together forcing the scFvs to dimerize), a triabody or a tetrabody (or an scFv with a linker peptide too short (optionally about one or two amino acids) for the two variable regions to fold together forcing the scFvs to trimerize or tetramize),
a single-domain antibody (dAB) (or a single variable region of an Ab heavy or Ab light chain),
a plurality of complementarity determining region (CDR) fragments, or
a multispecific antibody formed from two or more antibody fragments.

3. The recombinant antibody (Ab), or antigen binding fragment thereof, or monomeric or dimeric antigen binding protein, of claim 1,
wherein the antibody or dimeric antigen binding protein comprises a heavy chain variable region paired with or bound to a light chain variable region such that the antibody or the dimeric antigen binding protein is capable of specifically binding a human p40 (p63 isoform DeltaNp63, or ΔNp63) protein or polypeptide, an antigen or an epitope comprising an amino acid sequence MLYLENNAQTQFSEPQC-NH2 (SEQ ID NO:1).

4. The recombinant antibody (Ab), or antigen binding fragment thereof, or monomeric or dimeric antigen binding protein, of claim 1,
wherein the Ab, or antigen binding fragment thereof, or monomeric or dimeric antigen binding protein, comprises:
(a) a heavy chain variable region (VH) capable of specifically binding a human p40 (p63 isoform DeltaNp63, or ΔNp63) protein or polypeptide, an antigen or an epitope comprising an amino acid sequence MLYLENNAQTQFSEPQC-NH2 (SEQ ID NO:1), comprising:
(1) an amino acid sequence comprising the three CDR1, CDR2 and CDR3 complementarity determining regions (CDRs) of SEQ ID NO:1, or CDR1 amino acid (aa) residues GFSLSSYG (residues 25 to 32 of SEQ ID NO:2), CDR2 aa residues ISHITTT (residues 50 to 56 of SEQ ID NO:2), and CDR3 aa residues CRGQYGSGIIYAL (residues 94 to 106 of SEQ ID NO:2), or
(2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:2, or CDR1 amino acid (aa) residues GFSLSSYG (residues 25 to 32 of SEQ ID NO:2), CDR2 aa residues ISHITTT (residues 50 to 56 of SEQ ID NO:2), and CDR3 aa residues CRGQYGSGIIYAL (residues 94 to 106 of SEQ ID NO:2), or
(3) an amino acid sequence having at least about 70%, 75%, 80%, 85%, 90%, 95%, 98% sequence identity, or between about 70% to 100% sequence identity, to SEQ ID NO:2, or an amino acid sequence having complete sequence identity to SEQ ID NO:2;
(b) a light chain variable region (VL) capable of specifically binding a human p40 (p63 isoform DeltaNp63, or ΔNp63) protein or polypeptide, an antigen or an epitope comprising an amino acid sequence MLYLENNAQTQFSEPQC-NH2 (SEQ ID NO:1), comprising:
(1) an amino acid sequence comprising the three CDR1, CDR2 and CDR3 complementarity determining regions (CDRs) of SEQ ID NO:3, or CDR1 amino acid (aa) residues QSVYNNKN (residues 27 to 34 of SEQ ID NO:3), CDR2 aa residues YAS (residues 52 to 54 of SEQ ID NO:3), and CDR3 aa residues HGEFSCDSGDCSA (residues 91 to 103 of SEQ ID NO:3), or
(2) amino acid sequences having at least about 70%, 75%, 80%, 85%, 90%, 95%, 98% sequence identity, or between about 70% to 100% sequence identity, to each of the three CDR1, CDR2 and CDR3 complementarity determining regions (CDRs) of SEQ ID NO:3, or CDR1 amino acid (aa) residues QSVYNNKN (residues 27 to 34 of SEQ ID NO:3), CDR2 aa residues YAS (residues 52 to 54 of SEQ ID NO:3), and CDR3 aa residues HGEFSCDSGDCSA (residues 91 to 103 of SEQ ID NO:3); or
(3) an amino acid sequence having at least about 70%, 75%, 80%, 85%, 90%, 95%, 98% sequence identity, or between about 70% to 100% sequence identity, to SEQ ID NO:3, or an amino acid sequence having complete (100%) sequence identity to SEQ ID NO:3; or
(c) a heterodimer capable of specifically binding a human p40 (p63 isoform DeltaNp63, or ΔNp63) protein or polypeptide, an antigen or an epitope comprising an amino acid sequence MLYLENNAQTQFSEPQC-NH2 (SEQ ID NO: 1), comprising the heavy chain variable region (VH) of (a) and the light chain variable region (VL) of (b).

5. The recombinant antibody (Ab), or antigen binding fragment thereof, or monomeric or dimeric antigen binding protein, of claim 1,
wherein the heavy chain variable region comprises an amino acid sequence:
QSVEESGGRLVKPDESLTLTCTVSGFSLSSYGVTWVRQAPGKGLEWIGYI SHITTTYYASWAKGRFTISKTSPTTVDLKMTSLTTEDTATYFCCRGQYGS GIIYALWGPGTLVTISS(SEQ ID NO:2),
or
SEQ ID NO:2 having one or more amino acid substitutions or deletions, and the recombinant antibody (Ab), or antigen binding fragment thereof, or monomeric or dimeric antigen binding protein retains its ability to specifically bind to a peptide or epitope comprising SEQ ID NO: 1, or a fragment of a polypeptide of SEQ ID NO: 1, a p40 (or p63 isoform DeltaNp63) polypeptide or peptide or a fragment of a p40 polypeptide or peptide.

6. The recombinant antibody (Ab), or antigen binding fragment thereof, or monomeric or dimeric antigen binding protein, of claim 1,
wherein the light chain variable region comprises an amino acid sequence:
AQVLTQTPSPVSAAVGGTVTINCQASQSVYNNKNLAWYQQKPGQPPKLLl
YYASTLASGVPSRFSGSGSGTQFTLTISGVQCDDAATYYCHGEFSCDSGD CSAFGGGTEVVVK(SEQ ID NO:3),
or
SEQ ID NO:3 having one or more amino acid substitutions or deletions, and the recombinant antibody (Ab), or antigen binding fragment thereof, or monomeric or dimeric antigen binding protein retains its ability to specifically bind to a peptide or epitope comprising SEQ ID NO:1, or a fragment of a polypeptide of SEQ ID NO:1, a p40 (or p63 isoform DeltaNp63) polypeptide or peptide or a fragment of a p40 polypeptide or peptide.

7. The recombinant antibody (Ab), or antigen binding fragment thereof, or monomeric or dimeric antigen binding protein, of claim 1,
wherein the heavy chain variable region comprises an amino acid sequence:
QSVEESGGRLVKPDESLTLTCTVSGFSLSSYGVTWVRQAPGKGLEWIGYI
SHITTTYYASWAKGRFTISKTSPTTVDLKMTSLTTEDTATYFCCRGQYGS
GIIYALWGPGTLVTISS(SEQ ID NO:2)
, or SEQ ID NO:2 having one or more amino acid substitutions or deletions, and the recombinant antibody (Ab), or antigen binding fragment thereof, or monomeric or dimeric antigen binding protein retains its ability to specifically bind to a peptide or epitope comprising SEQ ID NO: 1, or a fragment of a polypeptide of SEQ ID NO:1, a p40 (or p63 isoform DeltaNp63) polypeptide or peptide or a fragment of a p40 polypeptide or peptide, and
the light chain variable region comprises an amino acid sequence:
AQVLTQTPSPVSAAVGGTVTINCQASQSVYNNKNLAWYQQKPGQPPKLLI
YYASTLASGVPSRFSGSGSGTQFTLTISGVQCDDAATYYCHGEFSCDSGD
CSAFGGGTEVVVK
(SEQ ID NO:3), or SEQ ID NO:3 having one or more amino acid substitutions or deletions, and the recombinant antibody (Ab), or antigen binding fragment thereof, or monomeric or dimeric antigen binding protein retains its ability to specifically bind to a peptide or epitope comprising SEQ ID NO: 1, or a fragment of a polypeptide of SEQ ID NO:1, a p40 (or p63 isoform DeltaNp63) polypeptide or peptide or a fragment of a p40 polypeptide or peptide.

8. The recombinant antibody (Ab), or antigen binding fragment thereof, or monomeric or dimeric antigen binding protein, of claim 1, wherein: the light chain variable region further comprises at least a portion of a light chain constant region,
wherein optionally the light chain constant region comprises and amino acid sequence:
GDPVAPTVLIFPPAADQVATGTVTIVCVANKYFPDVTVTWEVDGTTQTTG IENSKTPQNSADCTYNLSSTLTLTSTQYNSHKEYTCKVTQGTTSVVQSFN RGDC(SEQ ID NO:7).

9. (canceled)

10. The recombinant antibody (Ab), or antigen binding fragment thereof, or monomeric or dimeric antigen binding protein, of claim 1, wherein: the heavy chain variable region further comprises at least a portion of a heavy chain constant region,
wherein optionally the heavy chain constant region comprises an amino acid sequence:
GQPKAPSVFPLAPCCGDTPSSTVTLGCLVKGYLPEPVTVTWNSGTLTNGV RTFPSVRQSSGLYSLSSVVSVTSSSQPVTCNVAHPATNTKVDKTVAPSTC SKPTCPPPELLGGPSVFIFPPKPKDTLMISRTPEVTCVVVDVSQDDPEVQ FTWYINNEQVRTARPPLREQQFNSTIRVVSTLPIAHQDWLRGKEFKCKVH NKALPAPIEKTISKARGQPLEPKVYTMGPPREELSSRSVSLTCMINGFYP SDISVEWEKNGKAEDNYKTTPAVLDSDGSYFLYSKLSVPTSEWQRGDVFT CSVMHEALHNHYTQKSISRSPGK(SEQ ID NO:8).

11. (canceled)

12. The recombinant antibody (Ab), or antigen binding fragment thereof, or monomeric or dimeric antigen binding protein, of claim 1, wherein: the light chain variable region further comprises at least a portion of a light chain constant region; and, the heavy chain variable region further comprises at least a portion of a heavy chain constant region.

13. The recombinant antibody (Ab), or antigen binding fragment thereof, or monomeric or dimeric antigen binding protein, of claim 12, wherein the recombinant antibody (Ab), or antigen binding fragment thereof, or monomeric or dimeric antigen binding protein, comprises a light chain comprising an amino acid sequence (SEQ ID NO:9)
AQVLTQTPSPVSAAVGGTVTINCQASQSVYNNKNLAWYQQKPGQPPKLLI
YYASTLASGVPSRFSGSGSGTQFTLTISGVQCDDAATYYCHGEFSCDSGD
CSAFGGGTEVVVKGDPVAPTVLIFPPAADQVATGTVTIVCVANKYFPDVT
VTWEVDGTTQTTGIENSKTPQNSADCTYNLSSTLTLTSTQYNSHKEYTCK
VTQGTTSVVQSFNRGDC,
and
a heavy chain comprising an amino acid sequence (SEQ ID NO:10)
QSVEESGGRLVKPDESLTLTCTVSGFSLSSYGVTWVRQAPGKGLEWIGYI
SHITTTYYASWAKGRFTISKTSPTTVDLKMTSLTTEDTATYFCCRGQYGS
GIIYALWGPGTLVTISSGQPKAPSVFPLAPCCGDTPSSTVTLGCLVKGYL
PEPVTVTWNSGTLTNGVRTFPSVRQSSGLYSLSSVVSVTSSSQPVTCNVA
HPATNTKVDKTVAPSTCSKPTCPPPELLGGPSVFIFPPKPKDTLMISRTP
EVTCVVVDVSQDDPEVQFTWYINNEQVRTARPPLREQQFNSTIRVVSTLP
IAHQDWLRGKEFKCKVHNKALPAPIEKTISKARGQPLEPKVYTMGPPREE
LSSRSVSLTCMINGFYPSDISVEWEKNGKAEDNYKTTPAVLDSDGSYFLY
SKLSVPTSEWQRGDVFTCSVMHEALHNHYTQKSISRSPGK.

14. The recombinant antibody (Ab), or antigen binding fragment thereof, or monomeric or dimeric antigen binding protein, of claim 1, wherein the heavy chain constant region comprises amino acid sequence from a IgG, IgM, IgA, IgD or IgE isotype; or the light chain constant region comprises amino acid sequence from a kappa (k) or lambda (λ) isotype.

15. The recombinant antibody (Ab), or antigen binding fragment thereof, or monomeric or dimeric antigen binding protein, of claim 1, wherein the at least a portion of the heavy chain constant region, at least a portion of the the light chain constant region, or at least a portion of the heavy chain constant region and the light chain constant region, is or comprises amino acid sequence of a human, a rabbit, a mouse or a rat origin or comprises constant region amino acid sequence derived from a human, a rabbit, a mouse or a rat.

16. The recombinant antibody (Ab), or antigen binding fragment thereof, or monomeric or dimeric antigen binding protein, of claim 1, wherein at least a portion of the heavy chain constant region, at least a portion of the light chain constant region, or at least a portion of the heavy chain constant region and the light chain constant region, is or comprises a synthetic amino acid sequence.

17. The recombinant antibody (Ab), or antigen binding fragment thereof, or monomeric or dimeric antigen binding protein, of claim 1, or of any of the preceding claims, wherein the recombinant antibody, the antigen binding fragment thereof, or monomeric or dimeric antigen binding protein, or the heavy chain constant region, or the light chain constant region, or the heavy chain constant region and the light chain constant region, further comprises or is bound to a detectable agent or a binding moiety.

18. The recombinant antibody (Ab), or antigen binding fragment thereof, or monomeric or dimeric antigen binding protein, of claim 17, wherein the detectable agent or binding moiety is covalently conjugated to the recombinant antibody (Ab), or antigen binding fragment thereof, or monomeric or dimeric antigen binding protein.

19. The recombinant antibody (Ab), or antigen binding fragment thereof, or monomeric or dimeric antigen binding protein, of claim 17, wherein the detectable agent or binding moiety comprises a biotin, a fluorescent or chemiluminescent label, a fluorophore, sulfoindocyanine, nile red, rhodamine, perylene, fluorenyl, coumarin, 7-methoxycoumarin (Mca), dabcyl, [2-(4-nitro-2,1,3-benzoxadiazol-7-yl)aminoethyl]trimethylammonium (NBD), Nile blue, Tamra, boron-dipyrromethene (BODIPY), or derivatives thereof, a dye, a radioisotope, a quantum dot or photoluminescent aqueous nanocrystal, a hapten, or an antibody binding epitope or domain.

20. The recombinant antibody (Ab), or antigen binding fragment thereof, or monomeric or dimeric antigen binding protein, of claim 19, wherein:
(a) the fluorophore is or comprises a dansyl, a fluorescein, a carboxyfluorescein (FAM) or a 6-FAM moiety;
(b) the dye is or comprises a cyanine dye, a Cy3 or a Cy5; or
(c) the hapten is or comprises a biotin, a theophylline, a digoxigenin, a carborane, a fluorescein or a bromodeoxyuridine moiety.

21-22. (canceled)

23. A chimeric or recombinant nucleic acid comprising: a nucleic acid sequence encoding a chimeric or recombinant antibody or dimeric antigen binding protein as set forth in claim 1; or, a nucleic acid sequence comprising SEQ ID NO:2 or SEQ ID NO:3,
wherein optionally the chimeric or recombinant nucleic acid further comprises and is operatively linked to a transcriptional regulatory element,
and optionally the transcriptional regulatory element comprises a promoter,
and optionally the promoter is an inducible promoter or a constitutive promoter.

24. An expression cassette, a vector, a recombinant virus, an artificial chromosome, a cosmid or a plasmid comprising a chimeric or a recombinant nucleic acid of claim 23.

25. A cell comprising a chimeric or recombinant antibody or dimeric antigen binding protein of claim 1,
wherein optionally the cell is a bacterial, fungal, mammalian, yeast, insect or plant cell.

26. A method for detecting the presence of a human p40 protein in a cell, a tissue, an organ or a portion of any of the foregoing, comprising:
(a) contacting the cell, tissue or organ or portion of any of the foregoing with a chimeric or recombinant antibody or dimeric antigen binding protein of claim 1, and
(b) detecting binding of the chimeric or recombinant antibody or dimeric antigen binding protein with a p40 polypeptide or a MLYLENNAQTQFSEPQC-NH2 (SEQ ID NO:1)-comprising polypeptide in the cell, tissue or organ or portion of any of the foregoing,
thereby detecting the presence of a human p40 protein in a cell, a tissue, an organ or a portion of any of the foregoing, comprising contacting the cell, tissue or organ or portion of any of the foregoing,
wherein optionally the contacting comprises use of an immunohistochemistry (IHC) assay,
and optionally the method further comprises contacting the chimeric or recombinant antibody or dimeric antigen binding protein with a detectable agent to indicate or signal binding of the chimeric or recombinant antibody or dimeric antigen binding protein to the human p40 protein,
and optionally the detectable agent specifically binds to the chimeric or recombinant antibody or dimeric antigen binding protein.

27-29. (canceled)

30. A method for detecting or diagnosing a cancer,
wherein optionally the cancer is: a lung cancer, a lung squamous cell carcinoma, a lung adenocarcinoma or adenocarcinoma of the lung, a non-small-cell lung cancer, an adenocarcinoma, or a squamous cell carcinoma,
wherein the method comprises detection of expression or presence of a human p40 protein in a cell, tissue or organ sample, optionally in a cell, tissue or organ sample from an individual in need thereof, using a chimeric or recombinant antibody of claim 1, to detect the expression or presence of the human p40 protein in the cell, tissue or organ sample,
and detecting the expression or presence of the human p40 protein in the cell, tissue or organ sample detects or diagnoses the cancer,
wherein optionally the detection comprises conducting an immunohistochemistry (IHC) assay.

31. (canceled)

32. A method for treating, ameliorating or preventing a cancer comprising first detecting or diagnosing the cancer using a method of claim 30, followed by treatment of the individual in need thereof for the treatment, amelioration or prevention of the cancer,
wherein optionally the cancer is a lung cancer, a lung squamous cell carcinoma, a lung adenocarcinoma (adenocarcinoma of the lung), a non-small-cell lung cancer, an adenocarcinoma, or a squamous cell carcinoma.

33-37. (canceled)

38. A kit comprising a chimeric or recombinant antibody of claim 1,
wherein optionally the kit comprises components needed for an immunohistochemistry (IHC) assay.

39. (canceled)
